(19) United States
(12) Patent Application Publication
(10) Pub. No.: US 2024/0156566 A1
(43) Pub. Date: May 16, 2024

(54) CONTROL SYSTEM FOR ENCLOSURE GAS PRESSURIZATION, INFLATION, AND AIRFLOW MANAGEMENT

(71) Applicant: SURGIBOX INC., Cambridge, MA (US)

(72) Inventor: Mike Horia Mihail Teodorescu, Cambridge, MA (US)

(21) Appl. No.: 18/550,244

(22) PCT Filed: Mar. 11, 2022
(86) PCT No.: PCT/US22/20041

§ 371 Date: Sep. 12, 2023

Related U.S. Application Data
(63) Continuation of application No. PCT/US2021/058496, filed on Nov. 8, 2021.
(60) Provisional application No. 63/160,649, filed on Mar. 12, 2021.

(51) Int. Cl. A61B 90/40; A61B 90/00

(52) U.S. Cl. CPC A61B 90/40; A61B 2090/065; A61B 2090/401

(57) ABSTRACT

An enclosure-system includes an inflatable enclosure and a control system. The enclosure includes one or more enclosure walls made of flexible materials and at least one transparent section configured to allow users to observe the inside of the enclosure from the outside the enclosure. The enclosure includes one or more air vents, wherein at least one of the vents has a variable pneumatic resistance during operations. The control system is configured to control the environment inside the enclosure. The control system includes an air source configured to provide air to the enclosure, one or more sensors, and a processor.

### CROSS REFERENCE TO RELATED APPLICATIONS

This application claims priority from and the benefit of U.S. Provisional Patent Application No. 63/160,649 filed on Mar. 12, 2021 and International Patent Application number PCT/US2021/058496 filed on Nov. 8, 2021, which are hereby incorporated by reference for all purposes as if fully set forth herein.

### FIELD

This disclosure generally relates to control systems for controlling enclosure systems and the environments within enclosure systems, and to portable surgical systems and methods for regulating intra-operative environments over surgical sites.

### BACKGROUND

Inflatable enclosures and objects that include control systems have been used for various applications ranging from simple toy balloons to planetary devices. However, there is a need for control systems configured to monitor and control the dynamics of inflation processes, gas pressurization, pressures at various points in the enclosure, airflows, and other environmental parameters inside various types of enclosures (e.g. inflatable enclosures, rigid fixed volume enclosures). There is also a need that such control systems function as highly reliable and adaptive systems.

The above information disclosed in this Background section is only for enhancement of understanding of the disclosure.

### SUMMARY

This disclosure describes examples of an enclosure system for performing sterile operations. The enclosure-system includes an inflatable enclosure and a control system. The enclosure includes one or more enclosure walls made of flexible materials and at least one transparent section configured to allow users to observe the inside of the enclosure from the outside the enclosure. The enclosure includes one or more air vents, wherein at least one of the vents has a variable pneumatic resistance during operations. The control system is configured to control the environment inside the enclosure. The control system includes an air source configured to provide air to the enclosure, one or more sensors, and a processor.

The sensors include one or more pressure sensors configured to measure differential pressures between an interior of the enclosure and an exterior of the enclosure; one or more wall state sensors attached to or incorporated into the enclosure walls and configured to acquire wall state data representative of the straightening of the wall and the inflation level of the enclosure; and one or more airflow sensors configured to determine the airflow through the enclosure or through components of the enclosure.

The processor is configured to receive pressure data from the pressure sensors regarding the differential pressures and the dynamic evolution of the differential pressures. The processor is configured to receive wall state data from the wall state sensors, and to use the wall state data to determine an inflation state of the enclosure. The processor is configured to control the airflow provided by the air source and the air vents. The processor is further configured to control the pressure inside the enclosure and the airflow into the enclosure, based on information received from the sensors, so as to maintain the straightness of the walls above a predetermined minimum straightness ensuring good visibility through the walls, to maintain the inner enclosure pressure in a predetermined pressure range, and an airflow through the enclosure in a predetermined airflow range.

Some implementations of the disclosed subject matter include methods of operating the enclosure system. The methods include acquiring information from a plurality of sensors including pressure sensors, wall state sensors, and airflow sensors. The methods include processing the information acquired from the sensors and determining the pressures inside the enclosures, the airflows in the enclosure, and the straightness of the enclosure's walls. The methods include controlling the airflows in the enclosure, by controlling the air pump and vents/valves of the enclosure, according to the information received from the sensors.

Some implementations of the disclosed subject matter include methods for performing machine learning on the control processes associated with the inflation of the enclosure and the operation of the enclosure in a stationary normal operation regime.

It is to be understood that both the foregoing general description and the following detailed description are exemplary and explanatory and are intended to provide further explanation of the claimed subject matter.

### BRIEF DESCRIPTION OF THE DRAWINGS

One or more implementations of the subject matter described in this specification are illustrated in the accompanying drawings and described in the detail description below.

FIG. 1 shows a schematic diagram of an example enclosure system comprising a control system configured to receive information and to control an enclosure.

FIG. 2 shows an example enclosure system wherein the enclosure is part of a portable surgical system.

FIG. 3 shows an example air source configured to supply air and to control the airflow in an enclosure.

FIG. 4 shows an example enclosure system including an enclosure, an air source, an air duct or a flexible tube, pressure sampling tubes, and air vents.

FIG. 5 shows an example air source case that includes a protective cover configured to protect a filter of the air source from dust and other particles.

FIG. 6 shows an example adaptor configured to connect the air duct with the air source.

FIG. 7 shows example airflow separators and filter sensors.

FIG. 8 shows an example wall state sensor including a double wall.

FIG. 9 shows an example electronic system that receives and processes information received from multiple sensors.

FIG. 10 shows another example of the system shown in FIG. 9.

FIG. 11 shows an example implementation of the system in FIG. 9 that employs a different configuration of sensors, control systems, and alarms.

FIG. 12 shows a flow chart of an example method for monitoring the functioning of the pneumatic system formed by the enclosure, the air duct, and the control system.

FIG. 13 shows an example method of operating the enclosure system function of the differential pressure between the inside and the outside the enclosure.

FIG. 14 shows a flowchart of an example method for operating the enclosure system, for determining a pneumatic model of the enclosure, for acquiring knowledge about the enclosure, and for determining abnormal or faulty operations of the enclosure.

FIG. 15 shows several graphs illustrating examples of a control function configured to control the operation of the enclosure system.

FIG. 16 shows an example method of operating the enclosure system function of a plurality of physical parameters including pressure inside the enclosure, enclosure wall straightness, and airflows into the enclosure.

FIG. 17 shows an example method of operating the enclosure system that uses machine learning techniques and machine learned system models.

Like reference numbers and designations in the various drawings indicate like elements. Aspects of the disclosure are described with reference to the figures representing example implementations.

### DETAILED DESCRIPTION

The examples of enclosure systems described in this specification can be used for various applications, including when performing surgical procedures. For example, an enclosure system can be placed upon a patient when performing surgery to provide a clean and sanitized environment for the surgery and avoid contaminating the patient. The protection offered by the enclosure system can be vital particularly if the surgery is occurring in environments that may not be sufficiently sanitized. For example, when a surgical procedure is performed in a disaster zone or outside of a hospital, the environment surrounding the patient may be contaminated such that performing a surgery at that location may pose significant risks to the health of the patient due to environmental contamination. An enclosure system, such as the one described in this disclosure, can offer the patient protection. The enclosure system is carefully controlled through a control system that can control various control system environmental parameters such as pressure, airflow, temperature, gas type, and particulate/contaminant densities at various positions inside the enclosure and inside associated components of the enclosure. The control system can be configured to function with multiple types of enclosure systems and in multiple environments/circumstances.

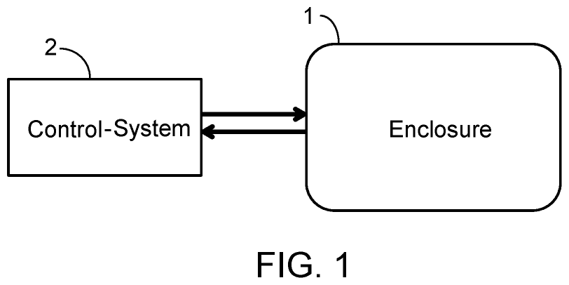

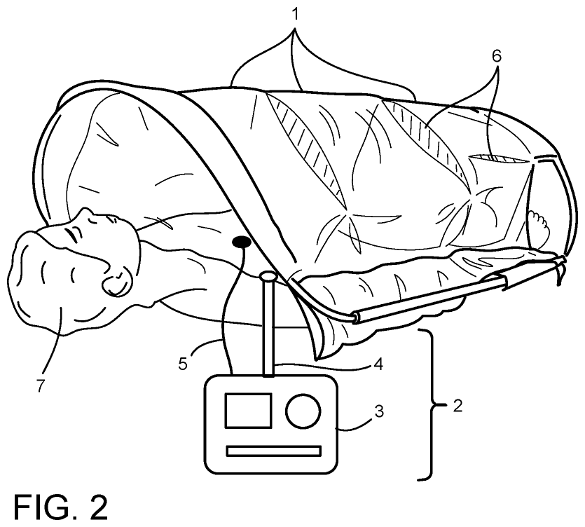

FIGS. 1 and 2 show a diagram of an example enclosure system. FIG. 1 shows a block diagram of the enclosure system, which comprises a control system 2 configured to transmit and receive information to and from an enclosure 1.

It is often desirable for enclosure systems to maintain a specified overpressure inside the enclosure with respect to the ambient and a specified minimal airflow through the enclosure so as to preserve a desirable environment inside the enclosure (e.g. desired air or gas purity and low levels of contaminants, undesired gases, or particulates). This disclosure describes implementations in which, an inflatable enclosure, a control system, and an environment control method implement an initial over-pressurization and gas circulation for an initial inflation of an enclosure 1 and subsequently sustain the over-pressure and gas flow through the enclosure 1.

The inflation and environment control methods may include controlling the dynamic process of inflation, controlling the airflows through the enclosure, preserving a specified overpressure, monitoring (or continuously monitoring) the conditions inside the enclosure and outside the enclosure. The monitoring may measure several parameters relating to the control methods and to the enclosure, as well as specific devices. The control system 2 may be configured to determine when the inflation and/or pressurization conditions are abnormal and to produce alarm signals. Because the operation circumstances and the controlled enclosures may vary, the control system 2 may be configured to have a high degree of self-adaptability and may include self-learning capabilities.

FIG. 2 shows an example implementation of an enclosure system. The enclosure system is part of a portable surgical system such as the one described in the international patent application number PCT/US2021/058496 titled “Portable System for Isolation and Regulation of Surgical Sites Environment,” which is incorporated by reference for all purposes as if fully set forth herein.

The enclosure system of FIG. 2 includes the enclosure 1 and the control system 2. The enclosure 1 is disposed over the body of a patient 7 such that one or more operators (e.g., a surgeon, a nurse, etc.) can access the torso of the patient 7, via an incise drape attached to the torso, and perform a surgical procedure on the patient 7. The enclosure 1 integrates arm ports 6 to allow access to the inside of the enclosure 1 by either operator arms or augmenting instrumentation taking the place of arms such as laparoscopes or robots. This way the operators can access and operate on the surgical site from inside the enclosure 1. The enclosure 1 may be supplied with air, via the control system 2, to form an inner sterile space/environment enclosed by the enclosure 1 above the operating-field, thereby enabling the user (e.g., a surgeon) to perform surgery in a sterile environment. The enclosure 1 may be supplied with filtered air under positive pressure. The control system 2 may include an air source 3, an air duct 4, and wire connectors 5 configured to enable the control system 2 to receive information from the enclosure 1 (e.g. sensor data from sensors disposed inside the enclosure 1) and to control various components of the enclosure 1 (e.g. valves). The air duct 4 may supply air from the air source 3 to the enclosure 1. The air duct 4 may be a flexible tube.

The control system 2 is configured to control gas pressurization, inflation, and airflow while also functioning as a highly reliable and adaptive system. The control system 2 is configured to determine a pressure difference between the inside and outside the enclosure 1, to adjust airflows in various parts of the enclosure 1, to generate a desired pressure or airflow dynamics, and to detect an anomalous operation (e.g., anomalous airflows and signal anomalies). The control system 2 is configured to perform machine learning by learning from previous control circumstances and control data including, e.g., the response time, the safety of the processes, the level of noise and vibrations (low levels desired), and power consumption (minimize power consumption).

The control system 2 may produce, control, and monitor the pressurization and/or inflation process of the enclosure 1. The control system 2 may be configured to maintain an overpressure and a specified minimal airflow in the enclosure 1 while monitoring elements of the overall enclosure system, such as: the enclosure 1, the coupling of the enclosure 1 to the control system 2, and a power supply. The control system 2 may be configured to generate the necessary signals and alarms and to display the relevant information. The control system 2 may have active or passive reservation blocks so as to ensure a high reliability in operation, capabilities of testing and creating a model of the enclosure 1, learning the model of enclosures, and diagnosing process abnormalities.

The control system 2 may include an electronic system that may further include one or more of: processors, displays, alarm devices, antennas, devices for wireless transmission of data from the sensors in or attached to the enclosure 1. The electronic system 20 may be configured to receive data from sensors in the enclosure 1, process the data, send control signals to various components of the enclosure system. Hereinafter, various components of the enclosure system are described.

Air Source Case

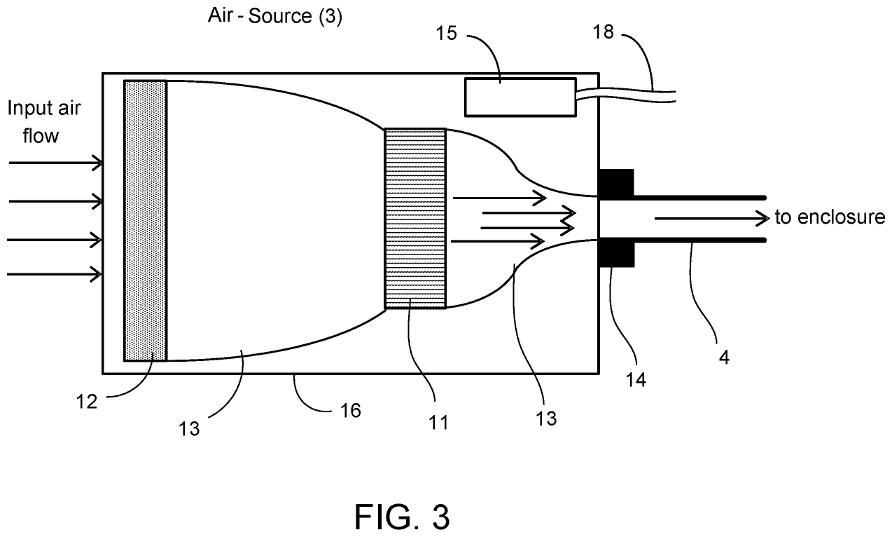

FIG. 3 shows an example implementation of the air source 3, which may include an air pump 11, an air filter 12, air circulating chamber 13, an adaptor 14 configured to connect with the air duct 4, an electronic subsystem 15, and a case 16 encasing the air pump 11, the air filter 12, the chamber 13, and the electronic subsystem 15. When the air pump 11 is in operation, the air source 3 may pull an input airflow from outside the case 16 into the case 16 through the filter 12, and direct the airflow into the air duct 4 and further into the enclosure 1. In some implementations, the airflow may enter through and be purified by filter 12. The airflow flow through a low turbulence coupling from filter 12 to the air pump 11 and is absorbed by the air pump 11, which creates a pressure at the output. The air flow may be expelled through a low turbulence coupling 14 into the air duct 4, and may be directed to the enclosure 1 via the air duct 4.

The air pump 11 may be a fan or other suitable pump for the volume of circulated air. A pre-filter may be provided in front of the filter 12 to protect the filter 12. A pressure sensitive valve may be included in the adaptor 14, at one end of the adaptor 14, or in another place along the airflow trajectory to allow the air circulation from the air pump 11 toward the enclosure 1 but to stop the reversed airflow from the enclosure 1 to the air pump 11. The air pump 11 and filter 12 may be arranged such that, relative to the air flow, in some cases, the filter 12 may be placed before the air pump 11, and, in some cases, the air pump 11 may be placed before the filter 12. The air pump 11 and filter 12 may be joined or placed close to each other such that no funnel-like junction between them is formed.

The air-circulating chamber 13 may be configured to ensure an essentially laminar airflow from the filter 12 to the air pump 11 and from the air pump 11 to the air duct 4 and the enclosure 1. The inner side of the walls of the chamber 13 may have surfaces that are specially treated or geometrically configured to reduce air friction. Chamber 13 may be funnel-like structures with wall configurations adaptable to the air velocity. For example, the walls may have, on the surfaces in contact with the airflow, scale-like elements that may be displaced and tilted with piezoelectric actuators, thus adapting the funnel shape towards reducing air friction with the walls.

The electronic subsystem 15 may include pressure sensors (e.g. differential pressure sensors) connected with one or more pressure sampling tubes 18. The sampling tubes 18 may sample the pressures at various places in the enclosure-system (e.g. enclosure 1, air duct 4, case 6) or in the ambient. The electronic subsystem 15 may further be connected to various devices and sensors of the enclosure-system via wires. The electronic subsystem 15, disposed inside the case 16, may be include into the overall electronic system 20 of the control system 2. The electronic subsystem 15 may be connected to sensors, displays, alarm devices, antennas, and devices for wireless transmission of data from the sensors in or attached to the enclosure.

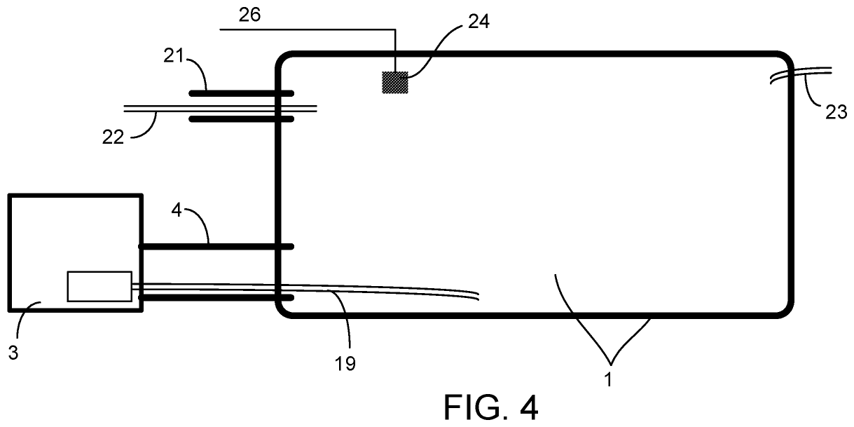

FIG. 4 shows an example implementation of an enclosure system that includes enclosure 1, air source 3, air duct 4, a first pressure sampling tube 19 disposed inside the air duct 4, an air vent 21, a second pressure sampling tube 22 disposed inside the vent 21, a third pressure sampling tube 23 directly coupled to the enclosure 1 protruding through the wall, and one or more sensors 24 disposed inside the enclosure 1. The sensors 24 may communicate with the electronic subsystem 15 via a wire 26 or by transmitting wireless signals. Sensors 24 may be disposed inside the enclosure 1 to monitor parameters such as: airflow, pressures, temperature, humidity, particulates levels, etc.

Filter-Cover

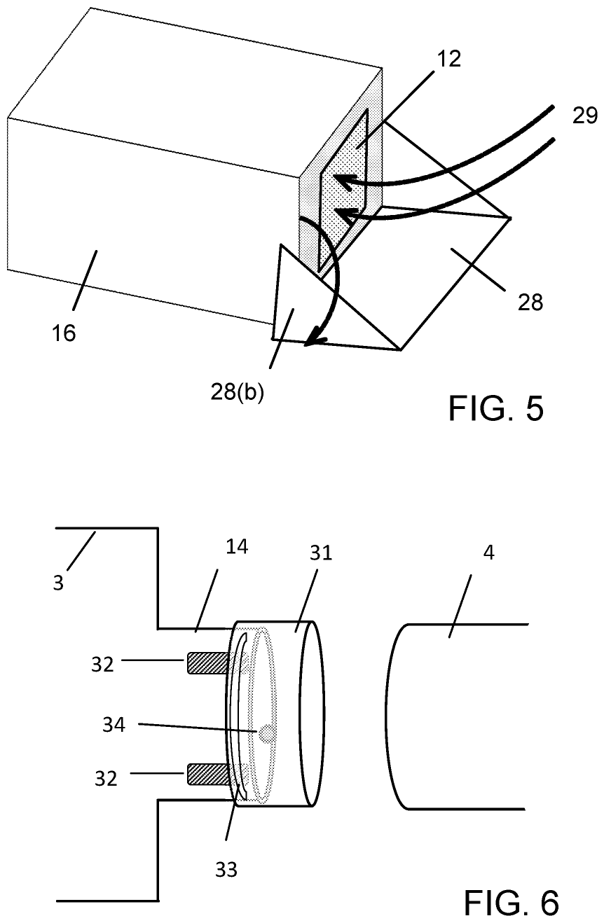

FIG. 5 shows an example implementation of a case 16 including a protective cover 28 configured to protect the air filter 12 from dust and other particles 29. This protective cover 28 may provide protection when the case 16 is placed directly on outside terrains or in industrial settings where the support surface may be covered with dust or industrial powders.

The pre-filter or the filter 12 may be protected during transportation or storage by a cover 28. The cover 28 may be foldable and may prevent dust and particles from the soil to be absorbed and enter the filter 12 or pre-filter. When opened, the cover 28 may form an angle of less than 90° with the vertical plan of the wall of the case 16 so that the suction flow comes mainly from upwards, thus preventing dust aspiration when the case 16 is positioned in the field on the ground. The cover 28 may include lateral wings 28(b) for preventing lateral aspiration of dust. A sensor may be provided to detect when the cover 28 is closed such that the operation of the air pump 11 is blocked when cover 28 is closed.

Adaptors & Connection Sensors

As shown in FIG. 6, the air source 3 may include an adaptor 14 configured to connect the air source 3 to the air duct 4 directing airflow (e.g. under pressure) from the air source 3 to the enclosure 1.

The adaptor 14 may include an airtight cap 31 configured to cover the adaptor opening so as to prevent contamination of the interior of case 16 when the air source 3 is not connected to the air duct 4. This may occur when the interior of the control system 2 and/or of the air duct 4 may be contaminated with particles, pathogen agents, or other hazardous elements from the ambient. The adaptor 14 may include one or more connection-sensors configured to determine if the adaptor is correctly connected with either the cap 31 or the air-duct 4. In some implementations, a connection sensor may include two metal electrodes 32 on the external surface of the adaptor 14, a cap electrode 33 on the inner surface of the cap 31 such that the cap electrode 33 shortcuts the electrodes 32 when the cap 31 is correctly connected, thus establishing an electric current path between the electrodes 32 on the adapter 14. A suitable electronic circuit detects the state of the electrical path between the two electrodes 32. When there is no current path between the two electrodes 32, an alarm is set to signal the contamination danger. Similarly, the air duct 4 may include a cap electrode 33 configured to work, in conjunction with electrodes 32, as a connection sensor.

In some implementations, a connection sensor may be a photodiode 34, a photo-transistor, or a similar device that is sensitive to light. When the cap 31 or the air-duct 4 are not covering a head of the adaptor 14, light may penetrate to the light sensitive device which then trigger an alarm. In some implementations, one set of connection sensors may be used for determining the correct connection of the cap 31 and another set of connection sensors may be used for determining the correct connection of the air duct 4. Different alarm signals can be generated when the cap 31 and/or air duct 4 are not connected properly (e.g., a first alarm signal in response to an improper connection of the cap 31 and a second alarm signal in response to an improper connection of the air duct 4) such that the control system 2 can differentiate the signals.

In some implementations, the connection sensors may include one or more of: capacitive electrodes forming a proximity sensor; two inductances constituting a proximity sensor; metal plates and an inductance constituting a proximity sensor; a pressure sensor (piezoresistive or other type); a small magnet and a Hall sensor forming together a proximity sensor; a photodiode or other light sensing device to sense the opening the opaque lid; or a combination thereof.

The control system 2 may use the information from the connection sensors to determine the time-periods (and time-lengths) during which the adaptor was open and exposed. In some implementations, if the control system 2 determines that the adaptor 14 was open for a period of time that is larger than a first threshold time period, a warning is issued. If a the control system 2 determines that the adaptor 14 was open for a period of time that is larger than a second threshold time period, the control system 2 may sound an alarm and/or stop operating and may decide that the air source 3 and/or the enclosure 1 have been contaminated. The control system 2 may also display a warning that the air source 3 and/or enclosure 1 are contaminated.

Filter-Sensors

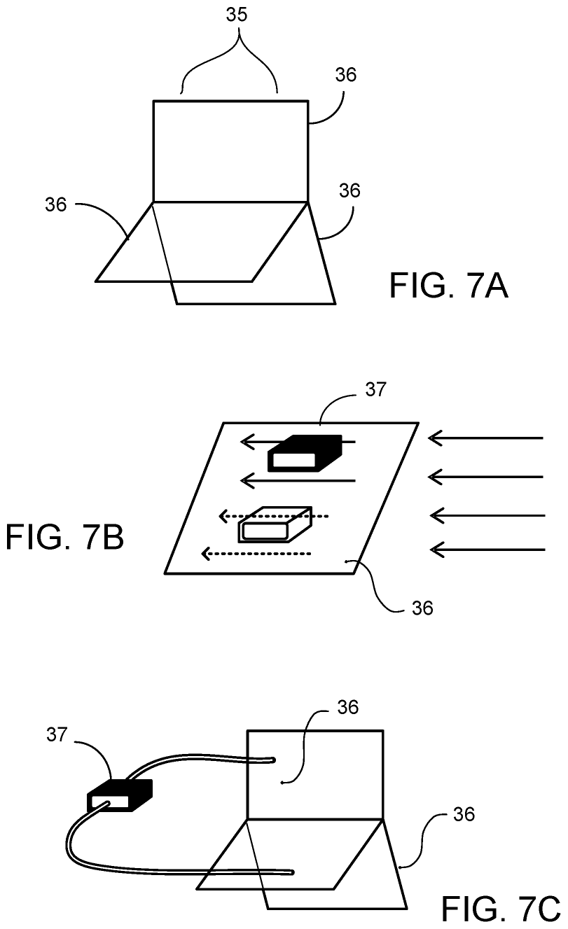

Referring to FIGS. 7(a) and 7(b), the air source 3 may include one or more airflow separators 35 disposed in the proximity of the filter 12 and dividing the airflow. The airflow separators 35 include one or more thin walls 36 disposed perpendicular on the filter 12. The airflow may be in a direction parallel to the walls 36 (as shown in FIG. 7(b). The airflow separators 35 may be disposed to form a honeycomb like structure. The airflow separators 35 may be configured to create desired laminar and uniform airflow, such as identical airflow in each cell of the honeycomb structure. The airflow separators 35 may be disposed on either side of the filter 12 or on both sides of the filter 12.

The air source 3 may further include one or more filter sensors 37 (as shown in FIGS. 7(b) and 7(c)) configured to provide information about air flows and/or pressures in different filter-areas. In some implementations, a filter-sensor 37 may measure a differential airflow and/or differential pressure between two adjacent filter areas separated by a plane 36 (as seen in FIG. 7(c)). The control system 2 may include a processor configured to receive information from the filter sensors 37, to determine a filter-status, and to generate a message about the filter status (e.g. filter functions well, filter needs to be changed, etc.) for display.

During operation, the air filter 12 may be clogged by dust and other particles or may even be perforated by larger particles or objects that may have moved with a large velocity. Therefore, to sustain the air filter operations, the air filtration can be monitored to detect problems similar to the ones described above. In some implementations, the uniformity of the filter operation is performed by comparing the dynamic and/or static pressure in sections of the airflow after or in front of the filter 12.

A method for monitoring filtration may include measuring the dynamic and/or static pressures (or differences between pressures/airflows) in symmetric regions of the airflow with a set of pressure sensors or a set of differential pressure sensors 36. Non-symmetries or temporal changes in asymmetry of the airflow may indicate potential filter clogging, which may occur asymmetrically up-down and center-to-sides, or even puncturing of the filter 12, which may be asymmetric, or other abnormality in the filter operation. To further increase the sensitivity of the method and of the measurement, thin walls (such as the walls 36 of the airflow separators 35) that do not obstruct the airflow and prevent the mixing of the airflows from different parts of the flow, may be placed symmetrically to separate symmetrical portions of the airflow. Then, differential measurements may be made between the regions separated by the thin walls. One planar or several semi-planar surfaces (walls) 36 can be used for this purpose. The filter-sensors may be pressure sensors and/or anemometers that sense the asymmetry of the velocities of the air flows in the respective regions.

The electronics of the control system 2 may use the information from the filter-sensors that detect asymmetries of the air pressure or airflow in the region behind the filter as delimited by the surfaces (walls) 36 and, when a substantial asymmetry is detected in the air pressure or flow, the control system 2 may generate warnings. The control system 2 may stop the operation when large asymmetries are detected, and may display (or use other means of announcement) a warning that the filter needs to be changed.

Pressure Sensors

For controlling the pressurization and/or inflation of an enclosure 1, either flexible and inflatable or of fixed volume, means for determining the pressure in the enclosure 1 and/or the static pressure difference between the inner part and the outside of the enclosure 1, and other means to detect the inflation state can be utilized. A variety of types of pressure and/or differential pressure sensors may be used, conditioned that their operating range, sensitivity, and response time satisfy the operation conditions for the intended application. The enclosure system may include one or more pressure sensors. The pressure sensors may include differential pressure sensors and/or absolute pressure sensors.

The pressure-sensors may be disposed inside the enclosure 1, such as the sensor 24 in FIG. 4, or may be disposed outside the enclosure 1 and connected to the inside part of the enclosure 1 through pressure sampling tubes such as tubes 19, 22, and 23 in FIG. 4. A pressure sensor disposed inside the enclosure 1 may send data to the control system 2 wirelessly or via wires connected to the electronic system 20.

The differential pressure sensors may be disposed such as to measure differential-pressures between an interior of the enclosure 1 and an exterior of the enclosure 1. In some implementations, at least some of the differential pressure sensors are attached to (or included into) enclosure walls and comprise a first measuring surface disposed inside the enclosure 1 and a second measuring surface disposed outside the enclosure 1 sense an ambient pressure. The differential pressure sensors are configured to measure a pressure difference between the inside and the outside of the enclosure 1.

Pressure Tubes & Vents

In some implementations, at least some of the differential pressure sensors are attached to pressure sampling tubes penetrating the enclosure walls. A differential pressure sensor may be disposed at (and connected with) one end of a pressure tube disposed outside the enclosure 1 whereas the other end of the pressure tube may be disposed inside the enclosure 1 to sample the pressure at a specific place inside the enclosure 1. Pressure sensors and pressure sampling tubes may be used to sample the pressures at various places in the enclosure system or immediately outside the enclosure system. The enclosure system may include several such tubes and sensors for measuring the pressure at different places of the enclosure-system. The pressure sampling tubes that traverse walls of the enclosure 1 may be coupled to airtight ports to prevent air leaks. A pressure sensor may be disposed inside the case 16 and may measure pressures outside the case 16, inside the enclosure 1, or inside the case 16 via pressure tubes as shown in FIGS. 2-3.

With reference to FIG. 4, the enclosure 1 may include one or more vents 21 such that air is continually circulated through the enclosure 1. The vents 21 may include valves configured to open the vent 21, close the vent 21, or adjust the pneumatic resistance of the vent 21. The air duct 4 may include one or more valves configured to open the air duct 4, close the air duct 4, or adjust the pneumatic resistance of the air duct 4. The processor 50 may be configured to control the airflow provided by the air source 3 and the airflow level through vents 21 and the air duct by controlling the pneumatic resistance of the valves (open, close, or intermediary resistance) coupled to the air source 3, the air duct 4, and/or the vents 21.

The vents 21 may include one or several pressure sampling tubes 22 that sample pressure inside enclosure 1. The vents 21 may also include airflow sensors. In some implementations, a pressure sampling tube 23 may enter the enclosure 1 through one of its walls through an airtight coupling. In some implementations, several differential pressure sensors are disposed inside the enclosure 1 and are coupled to pressure sampling tubes that sample ambient pressure. The differential pressure sensors may be included in the walls of the enclosure 1, with one side of the sensor exposed to the ambient pressure and the other side to the inner pressure, thus avoiding the use of pressure sampling tubes.

Processing Pressure Data

The pressure sensors may send data to the electronic system of the enclosure system wirelessly or through wires. The processor may be configured to receive pressure data from the pressure sensors regarding the differential pressures and/or the dynamic evolution of the differential pressures. The processor may use the pressure data in combination with data from others sensors to determine the inflation state of the enclosure 1. The electronic system 20 may be configured to receive data from sensors in the enclosure 1, process the data, and send control signals to various components of the enclosure system.

One or more sensors may be located inside the enclosure 1 and may measure one or more of air pressure, differential air pressure, air velocity, temperature, or other variables of the environment inside the enclosure 1. In some implementations, the sensors may send sensor data to the control system 2 through radio waves using antennas, such as strip antennas deposited on the walls or embedded in the walls of the enclosure 1.

The control system 2 may be configured so that a basic control of the system is established when the air duct 4 is connected to the air source 3 and the enclosure 1, the pressure sampling tubes are connected, and the control system 2 receives differential pressure readings.

Wall State Sensors

In case of flexible enclosures that are inflated, the pressure and differential pressure readings may be insufficient for a precise control of the state of the enclosure 1 and its walls. In fact, flexible walls may behave differently at different temperatures and may form wrinkles that are not tightened up at normal inflation pressure, creating excessive local stress in the walls or taking unsuitable forms when conditions vary. Therefore, an improved control of the pressurization should take into account the state of the walls too, in addition to the pressure and other physical parameters.

The enclosure system may include one or more wall state sensors configured to obtain data indicative of a state of the straightness of a portion of the enclosure 1's wall where the wall state sensor is placed and a level of inflation of the enclosure 1. The wall state sensors may be attached to the wall or may be incorporated into the wall at various positions of the enclosure 1. A processor 50 may be configured to receive the wall state data from the wall state sensors and to use the wall state data to determine an inflation state of the enclosure 1. The processor may be further configured to control the pressure inside the enclosure 1 and the airflow into the enclosure 1, based on information received from the sensors, thereby permitting the straightness of the walls to be maintained above a predetermined minimum straightness. Doing so ensures good visibility through the walls. In some implementations, the enclosure may have a maximum pressure value beyond which the pressure may pose a health risk to a patient within the enclosure. Thus, the control system 2 may control the pressure inside the enclosure 1 so that the pressure within the enclosure 1 is less than a pressure level associated with a patient safety threshold limit.

The wall state sensors may include one or more types of sensors, such as strain gauges, wall position sensors such as accelerometers along three axes (3D accelerometers), capacitive sensors, and inclination sensors. Such sensors may complement or replace pressure sensors in the assessment of the inflation state. The sensors for monitoring the state of the walls may be mounted inside the enclosure 1, attached to the walls, outside the walls, or may be included in the flexible walls. The control system 2 may further include one or more cameras configured to obtain still or moving images of the walls and to monitor the state of the walls. The processor may acquire images of the wall from the camera and may determine a state of the wall (e.g. wall straightness).

Double Wall Sensors

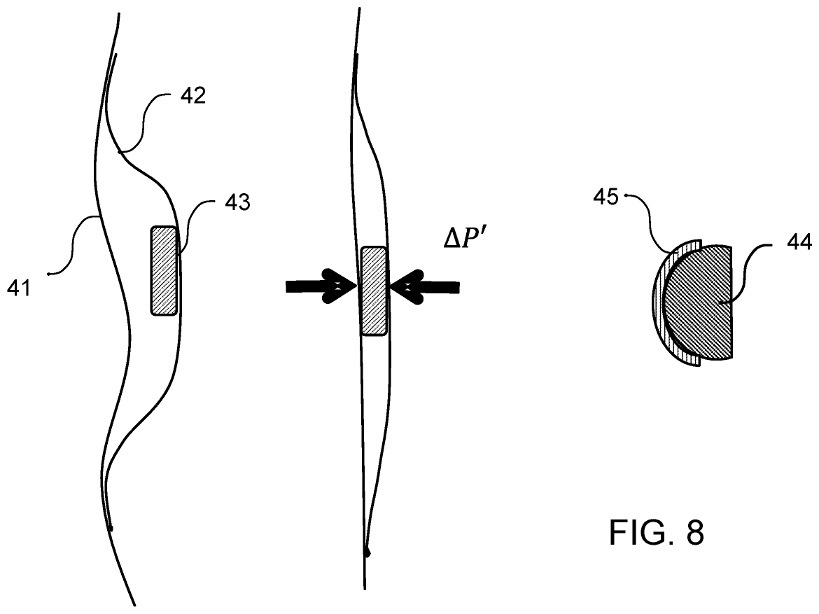

FIG. 8 schematically shows an example implementation of a double wall sensor. The double wall sensor may be disposed between a first wall 41 that may be a portion of the enclosure 1 wall and a second wall 42 that may be a flexible foil attached to the first wall 41 as seen in FIG. 8. The double wall sensor may include a force/pressure sensing device 43 that is disposed between the first wall 41 and the second wall 42. The first wall 41 and second wall 42 may form a small pocket within the enclosure wall. The first and second walls 41 and 42 may apply a compression force on the force/pressure sensing device 43 when the walls 41 and 42 are straightened due to, for example, a high differential pressure between the inside and outside of the enclosure 1. The force/pressure sensing device 43 is configured to measure the pressure/force generated by the walls 41 and 42 due to the straightening of the walls. When the enclosure 1 is fully inflated, the flexible wall 42 and the first wall 41 generate a pressure that is detected by the force sensor 43 and is indicative of the level of inflation of the enclosure 1. Thus, the force measured by force/pressure sensing device 43 provides a measure of the wall straightness. The force/pressure sensing device 43 may have any suitable shape including, e.g., a rectangular cross section, a circular cross section, or a cross section shaped as a circle segment 45 covering a pre-inflated bubble 44 that depresses as the pressure inside the enclosure 1 increases.

In some implementations, a semi-rounded object 44 may be placed in the pocket between walls 41 and 42 and may be covered by a pressure sensitive film 45, such as a piezoresistive film. The resistance of the film 45 changes in direct proportion to the straightening and tension in the walls 41 and 42. In such implementations, the object 44 with the piezoresistive layer 45 and the connections of the piezoresistive layer may form the sensor 43. Measuring the respective pressure signal is indicative on the straightening of the walls 41 and 42. In some implementations, the object carrying the piezoresistive film may have a parallelepiped shape with rounded corners, or the piezoresistive film may be attached directly to the enclosure wall or the flexible layer of the pocket. The processor may receive the wall state data from the force/pressure sensing device 43 and determine an inflation state of the enclosure 1 based on the wall state data.

Strain Gauge Sensor

A strain associated with the enclosure 1 can be detected using strain gauge sensors connected to the inner or outer surface of the walls of the enclosure 1. In some implementations, the wall state sensor may include a strain-gauge sensor disposed on and attached to the inner or outer surface of the enclosure wall and may deform together with the wall. The straightening/deforming of the wall may deform the strain gauge sensor according to the force proportional to a straightness level of the wall.

Cavity Sensors

In some implementations, a small sealed cavity may be created on the enclosure wall (inside or outside) by attaching a flexible layer on the enclosure wall. Thus, the cavity has at least one of its walls formed by the enclosure wall. The sealed cavity is pressurized slightly below the target pressure in the enclosure. A wall state sensor may be located within the sealed cavity. When the enclosure pressure reaches its target value, the cavity volume is reduced by the extension of the enclosure wall and by the internal pressure in the enclosure 1. The wall state sensor may include two electrodes forming a capacitor disposed inside the cavity. The electrodes of the capacitor move when the volume of the cavity is reduced, thereby changing the capacitance. The capacitance reaches a certain preset value when walls are straight.

Printed Interdigital Capacitor

In some implementations, to determine the straightening or inflation levels of a wall of the enclosure 1, an interdigital capacitor may be printed or disposed on a flexible support of the enclosure 1. The flexible support may be bonded to the wall and may deform together with the wall, thus modifying the capacitance of the interdigital capacitor. A certain value of capacitance is attained when the wall and the support attached are straight. The interdigital capacitor can be printed directly on the flexible wall of the enclosure 1.

Optical Fiber Sensor

In some implementations, the wall state sensor includes an optical fiber, a light source, and a fiber detector. The optical fiber is attached to (or incorporated into) a wall of the enclosure 1 and is configured to deform together with the wall. The deformation of the optical-fiber leads to a change in the signal sensed by the fiber detector.

The optical fiber becomes straight when the wall is fully distended and there is no light loss in the fiber, in which case the signal is maximum. When the wall is not straight, the optical fiber is bent together with the wall and, as a result of the bend, an optical loss occurs in the fiber. The signal detected by the fiber detector may provide an indication regarding how bent the fiber is and how straight the wall is. Further improvements in the measuring sensitivity and accuracy of the optical fiber sensor measurements, in addition adding the capability of measuring stress, are obtained by using optical fiber Bragg grating sensors or light polarizers with the optical fiber.

Various other methods of detecting the full distension of the walls, using other types of sensors, e.g., optical sensors, capacitive sensors, magnetic sensors, inductive sensors, resistive sensors, piezoresistive strain or stress sensors, piezoelectric sensors, mechanical deformation sensors, can be implemented. Also, image and video obtaining devices, including time-of-flight cameras, may be used to monitor the full distension and straightness of the walls.

The use of wall straightness or wall tension sensors positioned at one or several positions on the walls of the enclosure 1 is recommended because inner pressure sensors in the enclosure 1 offer an indirect, integrated information about the full and correct inflation of the enclosure 1. In some implementations, the sensor is an interdigital capacitive sensor that reaches a specific capacitance when the sensor support becomes planar.

In some implementations of the control system 2, where sensors for the straightness of the walls are used, information provided by the sensors for the straightness of the walls can be acquired by the processor of the control system 2 and is complementing and fused with the information from pressure sensors for improving the inflation control of the enclosure 1. For example, the control system 2 may store the pressure difference value between the inner side and the outer side of the enclosure 1 for which the wall is satisfactory distended, for a specific enclosure, and may utilize a target pressure difference for future inflation and pressurization processes, in a self-learning process of the control system 2.

Vibrations and Sound

The enclosure 1, the air duct 4, and the container of the control system 2 may be provided with sound and vibration sensors for controlling the amount of sound and vibrations generated during the inflation and pressurization processes. Other sensors, including flow sensors, dynamic pressure sensors, and temperature sensors may be used in the enclosure 1, in the control system 2 container, and in the air duct 4 from the control system 2 to the enclosure 1 may be used to further analyze the operating conditions and adjust the control accordingly.

Electronics

In some implementations, the electronics of the control system 2 may include a battery or other suitable power supply source, a circuit monitoring and regulating the charging and discharging of the battery, a voltage regulator, and a power supply monitor, one or several pressure and/or differential sensors, one or several sensors for airflow, wall distension sensors, sensors for the stress in the walls, temperature sensors, air humidity sensors, and sensors for other parameters of the inflated enclosure or ambient parameters useful in the control, one or several displays, one or several alarm blocks, a general use I/O interface, communication means, and core electronic system. The electronic system may perform a multitude of operations, including data acquisition, data processing, data storage, control function generation, generation of driving signals for the air pump, diagnosis and detection of abnormal conditions, generation of alarm signals, monitoring the entire system and the ambient, decision making on the control based on the monitored parameters, learning for improving control, and driving all the other output interfaces. One or more blocks of the electronic system may have reserves for increasing the reliability in operation.

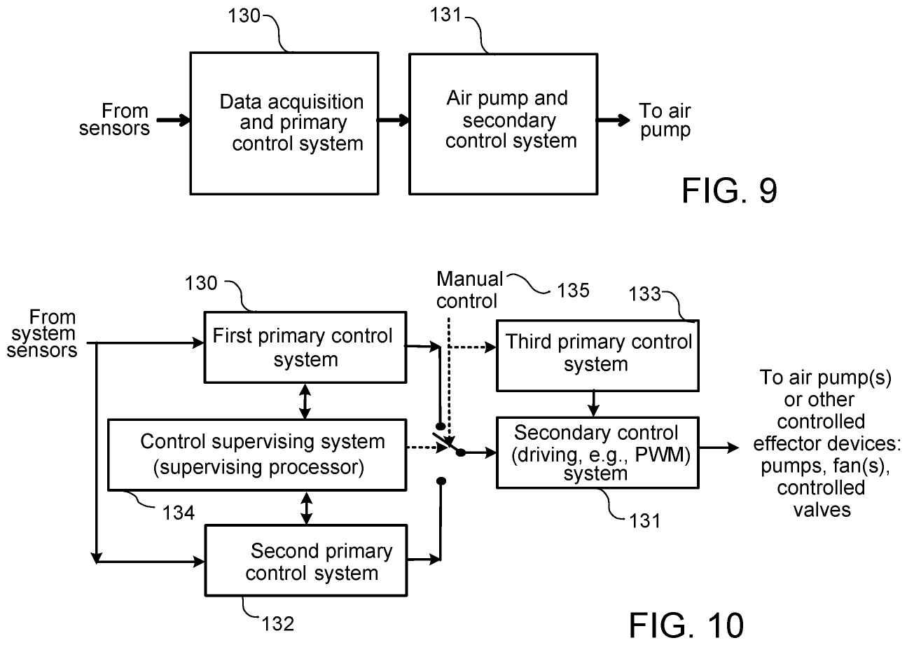

FIG. 9 shows a block diagram of an example implementation of an electronic system of enclosure 1 receiving input from one or multiple sensors, processing the input into a data acquisition and primary control system 130, and sending a control signal to an air pump control system 131. In some implementations, systems 130 and 131 may be one or more processors. In some implementations, systems 130 and 131 may be a combination of hardware and software and may include, e.g., circuits, such as first primary circuit 130 that converts signals received from the sensors into signals suitable for processing by the secondary control circuit 131, which in turn may drives the air pump.

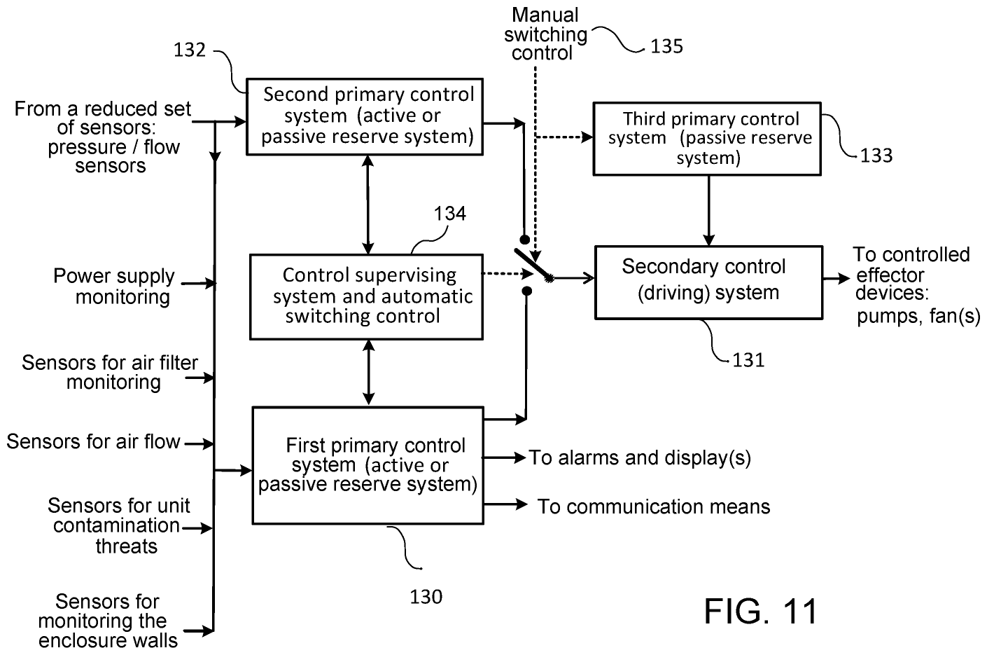

FIGS. 10 and 11 show example implementations of the system represented in FIG. 9 including a primary control system 130, a second primary system 132, a supervisor processor 134 which oversees the proper operation of systems 130 and 132 and a manual control switch 135 which the user can set to a third completely separate backup primary control electrical system 133, which may send a signal to a driving electrical system 131.

The second primary control block 132 may provide a redundancy in the operation of the first primary control system 130, and may have a second reserve, the third primary control circuit 133 configured to be used for critical applications. The first primary control block 130 is digital, while the second primary control electric circuit 132 may have reduced functionality for graceful degradation operation and enhanced reliability (e.g., if an integrated circuit in the first primary control block 130 fails to function properly, a backup circuit in the second primary control electric circuit 132 may operate that may have acceptable performance but may not match the performance standards of the integrated circuit in the first primary control block 130), and may use some analog circuits to convert the signals from the sensors into signals suitable for the input to the secondary control circuit 131.

The secondary control circuit 131 may receive the input signals from any of the primary control electrical systems and may convert the received signals into signals for driving an air pump. The primary control systems 130, 132, 133 may also perform parametric monitoring of the operation of the air pump, of the airflow through the pneumatic system, and of the battery. The primary control systems 130, 132, 133 may produce visual and audible alarm signals, with any of the primary control systems 130, 132, 133 also displaying information on the operation and suitable warning texts and operation suggestions on a display. Switching between the first and second primary control systems is produced either manually or automatically by a supervising system 134 that monitors the operation of the first primary control system 130 and detects its failure. The switching to the third primary control system 133 can be made manually, with the switch control 135, for maximal control of the operation by the human operator.

The primary control systems 130, 132, 133 may perform specific signal processing tasks including filtering signals from noise, smoothing the signals from the sensors, and fusing the information from the various sensors (e.g. fusing information from pressure sensors with information from the wall state sensors).

The first and second primary control systems 131, 132 may produce either analog or digital signals to control the secondary control system 131. The supervising system 134 may monitor the state of operation of the first primary control system 131, decide to switch operation to the second primary control system 131 when the operation of the first primary control system 130 is deficient, and decide when to reset the operation of the first primary control system 130.

One or more sensors may have analog/digital outputs and may be connected to the electronic system or may transmit wireless (optically, ultrasonically, using radio electromagnetic waves, infrared or other suitable means) the information to the electronic system.

Part of the electronic system may be digital, when the sensors produce digital outputs, or may be a hybrid analog and digital system when at least some of the sensors have analog outputs. The data acquisition and primary control system may be fully analog when functions such as supervision, fault identification and other complex functions are not required or are reduced to simple functions that can be easily implemented with analog circuits.

While the separation of the functions into primary and secondary control systems 130-133 is one example implementation utilized because it is useful for programing purposes and simplified design, in some implementations, one or more physical devices, such as a microcontroller or FPGA, may implement the functions of both the primary and secondary control systems 130-133. In some implementations, the second data acquisition and primary control block may be fully analog when functions such as supervision, fault identification and other complex functions are not required or are reduced to simple functions that can be easily implemented with analog circuits.

The operation of the control system 2 in a specific application may be dependent on the criteria set forth for the system optimization. For instance, in some implementations, the criterion may be safety operation. Another criterion may be maintaining a constant pressure in the enclosure, which can be easily implemented for embodiments where the minimal airflow criterion or a combination of criteria are used for optimization.

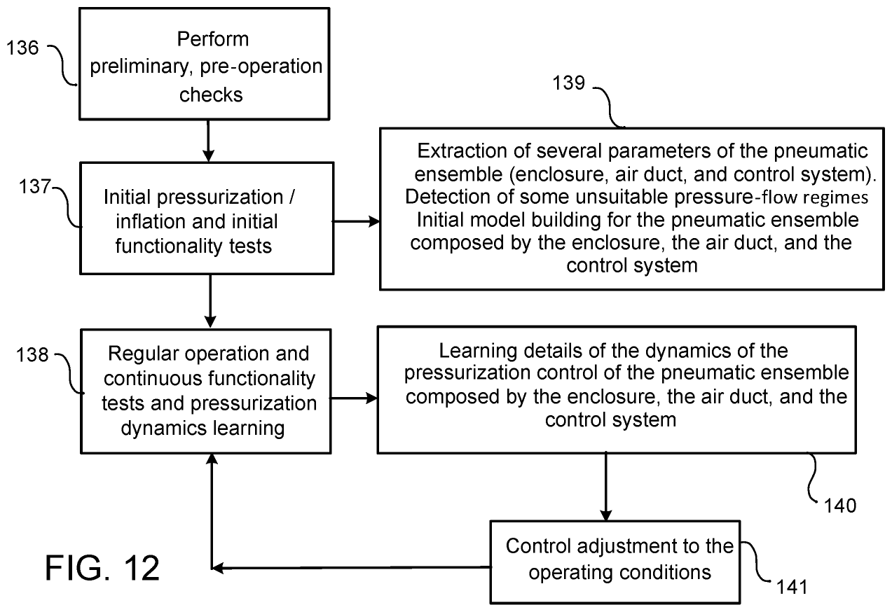

FIG. 12 shows a flow chart of a method for monitoring the functioning of the pneumatic enclosure system including the enclosure 1, air duct 4, and control system 2 (including the air source 3). In some implementations, the control system 2 may start by performing preliminary, pre-operation checks as shown in step 136. When power is turned on, the electronic system of the enclosure 1 acquires signals from one or several sensors and initiates the analysis of the setting and operation of the enclosure system. At this stage, the enclosure system may determine if the connection of the air duct 4 to the air source 3 and the enclosure 1 is correct, by receiving signals from the connection sensors. Then, the system may check whether the filter air cover 28 is open.

In some implementations, initial functionality tests as shown in step 137 (and preliminary operation checks) are performed for determining the functionality of the enclosure system. The functionality of the enclosure system is tested by initializing pressurization/inflation with linear (stepwise, staircase) or other predefined function of the differential pressure and applying the control for increasing the differential pressure for a specified time to. During this stage, the symmetry of the airflow behind the air filter 12 may be checked, warnings may be generated when the symmetry is not satisfactory, and the operation may be stopped for large asymmetries. Also, during this stage the airflow may be determined for the air source 3 and/or the pressurized enclosure 1. The airflow may be compared with predetermined values or with values learned from the operation of similar enclosures, and warnings may be generated when significant discrepancies occur. In addition, vibrations and sound produced in the enclosure 1, its walls, in the air duct 4, or in the container of the control system 2 during inflation are monitored for learning and then avoiding specific control parameters causing unwanted behaviors (e.g. too much noise and/or vibrations). The control parameters may include air pump power/airflow output and status/pneumatic resistance of various valves. During this phase, knowledge acquisition and model building as shown in step 139 are performed. Several performance parameter values of the enclosure system are extracted, such as the approximate flow resistance of the exhaust from the enclosure 1 and the approximate volume of the enclosure 1. Unsuitable pressure flow regimes that produce large vibration and noise levels are stored for avoiding them during normal operation.

The control method of FIG. 12 includes a pre-test health check of the system step 136; an initial pressurization and functionality tests step 137, a functionality test block and dynamic pressurization learning block 138, a measurement and learning block 139 which measures parameters of the pneumatic ensemble and initial model learning for the pneumatic system formed by the enclosure, the air duct, and the control system; and a regular operation block 138, which after initial learning of the performance parameter values monitors 140 the functionality and pneumatic system parameters in a continuous loop and adjusts, as necessary, controls to the operating conditions in step 141. Steps 138-140-141 operate in a loop under normal operation conditions for the enclosure system to continuously monitor the parameters of the system and make adjustments to the operating conditions as needed.

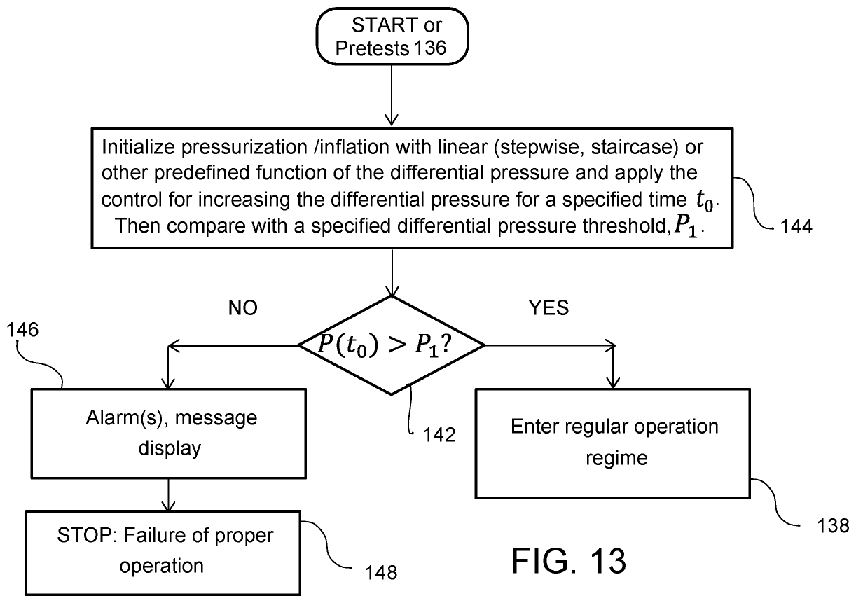

FIG. 13 shows a control method that includes the operations of running a set of pre-initialization tests (block 136), initializing pressurization of the enclosure 1 with a predefined function (block 144), and testing whether a specified pressure condition 142 has been met within a specified time (block 142). If the condition 142 has not been met in the specified time, then the control system 2 issues an alarm and enters a failure mode (blocks 146, 148). If the condition 142 has been met in the specified time, the control system 2 enters normal operation regime as shown in block 138 in FIG. 12.

During the initial pressurization stage (block 144), the enclosure system may compare the actual differential pressure between the inner and the outer side of the enclosure 1 with a specified differential pressure threshold P₁. When the differential pressure obtained is not greater than the threshold P₁ for a certain time period since the initiation of the pressurization as shown in block 142, the enclosure system may generate alarms and display a corresponding message on the display (block 146). The enclosure system may stop its operation until the system is restart after remedial measures have been taken to address the pressure (block 148). If the initial inflation or pressurization tests generate a positive outcome, the enclosure system may execute its regular operation regime (block 138). The regular operation regime may include various operations including, for example, electrical noise filtering in the signals from the sensors. For example, low pass analog filters may filter signals provided by the analog sensors and/or digital filters. Low-pass filtering can be used for the signals received from the pressure, airflow and wall sensors used with flexible enclosures because low-pass filters may remove variations of the signals due to movements of the walls of the enclosure 1 under external forces, e.g., human manipulation, wind. A further improvement of the quality of the signals is obtained by median filtering or other statistical filters adapted to the uses of the enclosure 1.

In some implementations, the system performs additional initial tests, including the measurement of the dynamic pressure and/or air velocity at the enclosure exhaust and/or in the air duct 4, and compares these dynamic pressures with stored values and/or with the airflow at the exist of the control system 2, for determining if air leaks occur or if the exhaust or the air duct 4 is blocked. A warning and/or alarm may be triggered when the conditions in the initial tests are not consistent with the desired operating conditions.

In some implementations, after the tests of the preliminary (pretest) phase and the initial phase, in which the initial phase includes the inflation if the enclosure 1 is flexible, the pressurization control system enters the regular operation regime 138 to maintain a constant differential pressure. This regular operation regime 138 may be based on the reading of the differential pressure between the enclosure 1 and the ambient environment surrounding the enclosure 1 with the aim of maintaining constant differential pressure. During the regular phase operation, the enclosure system may learn details of the dynamics of the pressurization control of the enclosure 1, the air duct 4, and the control system 2 using specific learning algorithms 40. The learning may be supplemented by providing the control system 2 with training data obtained from an operator or other enclosure systems. The acquired knowledge is used in the future adjustments of the control to the operating conditions, in step 141.

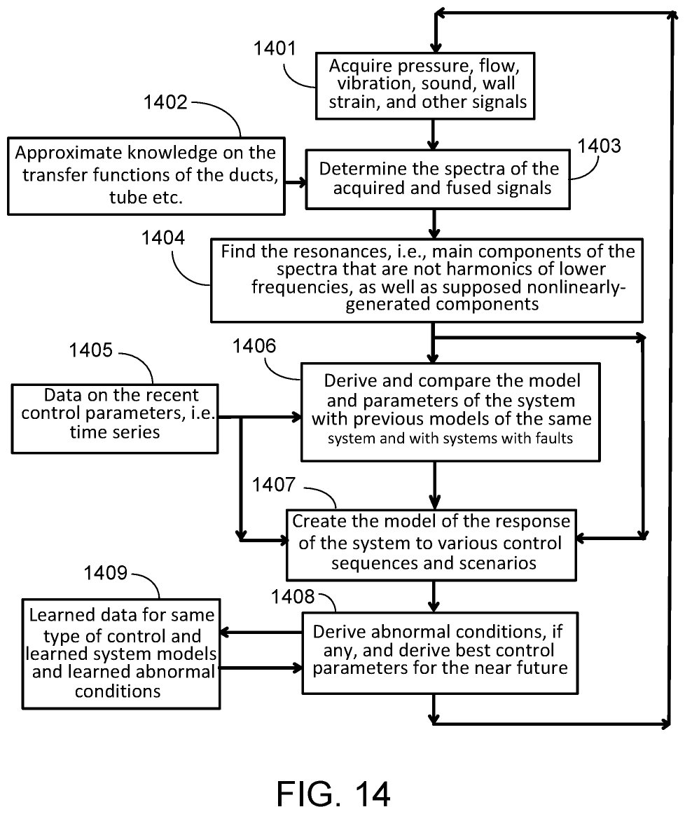

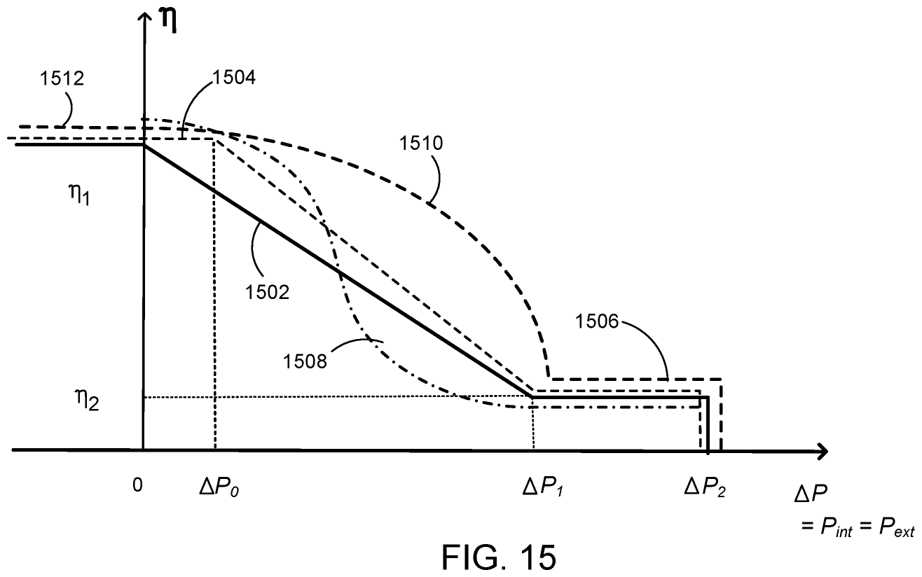

FIG. 14 shows a flowchart of a method for operating the enclosure system for determining a pneumatic model of the enclosure 1, for acquiring data about the state or condition of the enclosure 1, and for detecting abnormal conditions or operations.

The method may include an operation 1401 in which the control system 2 acquires data and information from the sensors about one or more of the following: pressures and air flows inside the enclosure 1 and other components of the enclosure system (e.g. vents 21, air source 3); sounds generated by the enclosure system; vibrations of the enclosure system; and wall strain. The method may further include an operation 1402 in which the control system 2 acquires data and information about transfer functions of the air ducts 4, vents 21, and valves of the enclosure system.

The method may further include an operation 1403 in which the control system 2 uses the information acquired at operations 1401 and 1402 to determine a series of properties of the acquired signals and data, such as a spectra of the signals. The information may include fused data and/or signals of two of more data/signals, such as pressure data and vibration data. The method may further include an operation 1404 in which the control system 2 determines resonances of the enclosure system, such as the main vibrational components of the spectra, vibrational components of the spectra which are not components of lower frequencies, and non-linearly generated components.

The method may further include an operation 1405 in which the control system 2 acquires and saves data associated with recent control parameters, such as time series for the control parameters. The method may further include an operation 1406 in which the control system 2 uses information generated at operations 1404 and 1405 to derive and compare a model of the system and parameters of the system with previous models of the same system and with models corresponding to various faulty operation regimes.

The method may further include an operation 1407 in which the control system 2 uses the information generated at operations 1404, 1405, and 1406 to create models for the response of the control system 2 to various control sequences and scenarios.

The method may further include an operation 1408 in which the control system 2 performs operation analysis on the entire system and determines if any abnormal operation conditions (e.g. faulty operations) are present or if the system operates properly. The operation analysis is based on the information generated at operation 1407 and/or on machine-learned data generated at operation 1409.

The method may further include an operation 1409 in which the control system receives data generated at operation 1408 regarding the functioning of the system, performs machine learning on the data, develops and saves learned data for the same type of control, develops and saves learned system models, and develops and saves learned abnormal conditions and operation regimes. The operations 1401 to 1409 may be repeated in a loop and performed iteratively.

The operations of method shown in FIG. 14 may be repeated iteratively or periodically to obtain measurements on the enclosure system at various times (in a first time ordered series t₁, t₂, t₃, . . . tₙ, n being any number greater than 1) and acquire a time ordered series of physical parameter measurements of the enclosure system. The measured physical parameters may include one or more of the following: enclosure pressures, airflows in the enclosure system, wall status, vibrations of the enclosure system, and sound generated by the enclosure system.

Based on information regarding the physical configuration of the enclosure system, the control system may generate one or more physical models for the enclosure system. At least one of the physical models may describe the vibrational behavior of the enclosure system.

The control system may control and set the enclosure system according to a time series of control-parameters (e.g. at times in a second time ordered series T₁, T₂, . . . Tₘ). The set control parameters may include a pulse controlling the duty-cycle of the air pump, an output airflow generated by the pump, any other parameters controlling the air source. The control system 2 may further control the pneumatic resistance of one or more of the enclosure system parts, e.g., vents, the air ducts, and air output ports by controlling valves included in the vents, air ducts, and air ports. Each of the controlled valves is associated with one or more control parameters (e.g., valves parameters).

The control system 2 may determine status of the enclosure system, based on the physical parameters and the control parameters, and whether the status corresponds to faulty operation or proper (e.g., non-faulty) operation. The status of the enclosure system may include at least a vibrational status, a pneumatic status, and an airflow status.

The control system 2 may determine a time series of control parameters leading to proper (e.g., non-faulty) operation of the enclosure system and set the control system 2 according to the time series of the control parameters. The time series of the control parameters may be determined as function of the status of the enclosure system, the physical models, and one or more machine-learned system models.

The control system 2 may be configured to generate a system model based on the status of the enclosure system, the physical models, and one or more machine-learned system models. Then the control system 2 may update the machine-learned system models with the generated system model.

For a specified air pump, there is a pump regulating parameter that determines the operating point on the pressure-airflow characteristic of the pump, with the airflow increasing when that parameter increases. For example, for an air pump driven by an electric motor, the increase of the duty cycle of the pulse width modulation (PWM) controlling the pump increases the airflow at a fixed pressure over the pump. Therefore, the increase in the regulating parameter, for example PWM duty cycle, determines an increase of the pressure difference between the enclosure 1 and the ambient (e.g., outside the enclosure system), in a manner dependent on the pressure-flow characteristic of the pump. The control system 2 may include a signal conditioning and primary control law generation sub-system and a converter sub-system that converts the primary control law into the PWM control law or other control law specific for the type of motor or other actuator used to perform the physical level operation of air pumping and air pressure control, such as valves.

In some implementations, the regulating parameter (also referred to as a control parameter) is the PWM duty cycle it and may be used to maintain a constant differential pressure ΔP between the interior and the exterior of the pressurized enclosure. The pumping-power is another control-parameter, related to the PWM duty cycle η, which may be used hereinafter.

A method of operating the enclosure system may include the operations described hereinafter. The control system 2 may determine a differential-pressure or a sequence of differential pressures ΔP at various times. The control system may evaluate a control-function F(ΔP) having input arguments including the differential-pressures ΔP and returning one or more values of control parameters, such as airflow output value to be generated by the air pump. The control function F is configured to return control parameters values for operating the enclosure system according to a desired operation regime. The control system 2 may adjust the control parameters (e.g. output of the air pump) to the value returned by the control function for the measured differential pressures.

FIG. 15 shows graphs of several control functions F for a range of differential-pressures ΔP where the returned control parameter is the PWM duty cycle η of the air pump. In some implementations, the control system 2 may be operated according to the control-function 1504 as described in the following. For differential-pressures ΔP smaller than ΔP₀, the returned parameter η has a constant value η₁ (e.g. which is a relatively large value, close to a maximum value) thereby resulting in a large pump airflow-output. For differential-pressures ΔP larger than ΔP₀ and smaller than ΔP₁, the returned parameter r) decreases linearly from η₁ to η₂. The value of η₂ is relatively small and may correspond to an airflow during the stable/normal operation range when some airflow through the enclosure is desirable. For differential-pressures ΔP larger than ΔP₁ and smaller than ΔP₂ the returned parameter η is kept constant to η₂ value. For differential-pressures ΔP larger than ΔP₂ the returned parameter η is zero, which corresponds to zero air-flow provided by the pump.

The control function F may include a set of adjustable parameters to configure the shape of the control function. The adjustable parameters may include one or more of the following: pressure values such as ΔP₁ and ΔP₂; trigger control parameters such as η₁ and η₂; and linearity/nonlinearity coefficients. The control-function F may be designed to generate various inflation regimes, depending on circumstances, such as, e.g., fast inflation, slow inflation, and high degree of control inflations, etc.

A simple relationship that satisfies the condition that an increase in the regulating parameter determines an increase of the pressure difference (and the same in terms of decrease) is a linear function ΔP=a+b∂. The control function F=η(ΔP)) may include a linear region as shown by 1502 and 1504. The linear dependence may be limited at ΔP=0 by a maximal value of η=100% and may extend up to some pressure difference equal or slightly larger than the desired pressure difference ΔP₀. Several variations of the linear law are directly derived, for specific applicative cases. As an example, when the enclosure should be fast pressurized, a large value of η, close to 100%, may be chosen for ΔP=0, conditioned that such a fast pressurization has no disadvantages, such as large levels of noise generated, or too high power consumed. Also, when the enclosure 1 is to be fast pressurized, the same high value of 1 may be maintained in an interval [0, ΔP₁], as shown in 1504. When the enclosure 1 includes a vent and a minimal flow is to be ensured throughout the enclosure 1, a minimal value of η, η₂ may be maintained constant, as shown in 1506, in an interval [ΔP₀, ΔP₂], or q may be decreasing from η₂ to 0 in the same interval. Nonlinear relationships η=η(ΔP), such as 1508 and 1510, may be adopted.

According to some implementations, the enclosure system may initially start with a default, linear law or control-function F=η(ΔP) that suits the design constraints, for example maximal power consumption and maximal level of emitted noise, then continually learns to improve the control law according to a specified criterion or set of criteria.

The control function F can be modified over time to slowly decrease 1 from the value q(ΔP₂) to zero at some maximal pressure difference ΔP₃, for example according to a linear law. Also, a smoother control function can be used, that has in the entire control interval a continuous derivative. Also, a negative pressure difference may occur at the beginning of the inflation of inflatable enclosures if the enclosure was packed and sealed at a higher altitude and thus the pressure inside it is below the atmospheric pressure at the inflation place. Consequently, the system should respond to pressures ΔP<0, as shown by graph region 1512.

In addition to the monitoring of the differential pressure, the control system 2 may obtain repeated measurements and monitoring of other physical parameters, such as airflows and temperatures, and may adjust the control function accordingly. In addition to the control tasks, the control system 2 may monitor the state of the battery when a battery is used to power the system and may produce warnings when the battery level is low (e.g., an alarm when the battery level is below a threshold level).

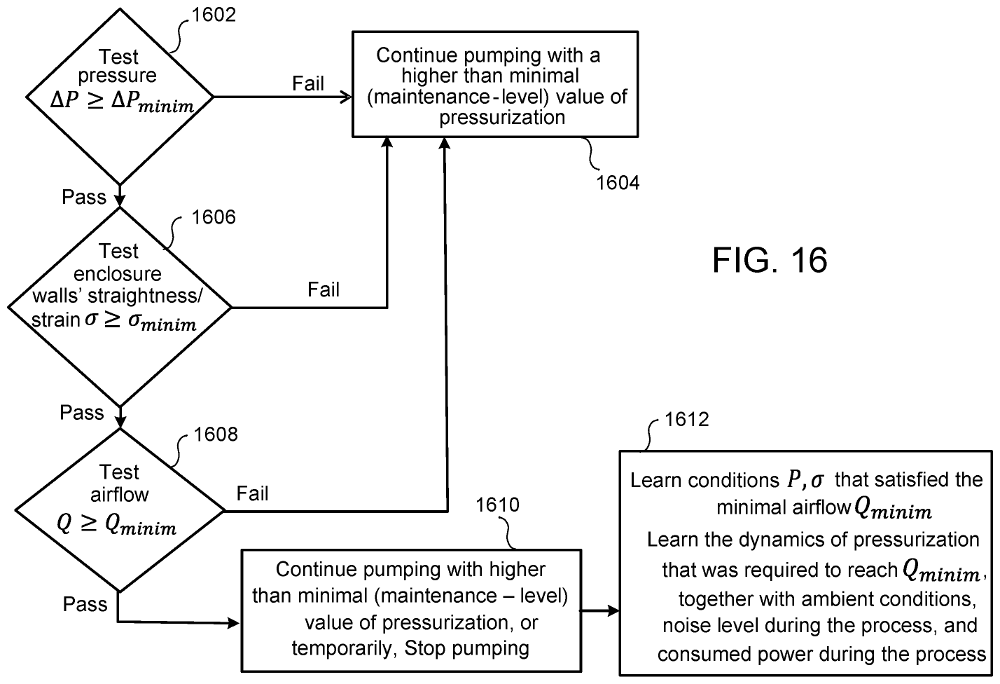

A method of operating the enclosure system is described hereinafter with reference to FIG. 16. The control system 2 may continuously test the conditions that determine the control regime and parameters by performing the following operations. First operation (1602): the control system 2 may determine if a pressurization condition ΔP≥ΔPₘᵢₙᵢₘ is satisfied (where ΔP is the differential-pressure between the inside and the outside of the enclosure 1, and ΔPₘᵢₙᵢₘ is a minimum-pressure threshold). Second operation (1606): the control system 2 may determine if the full distension of the walls condition (enclosure walls' straightness or strain) σ≥σₘᵢₙᵢₘ is satisfied (where σ is a measure of wall straightness and σₘᵢₙᵢₘ is a minimum wall straightness threshold). This step is valid for inflatable enclosure, it is missing when the enclosure 1 is rigid (fixed volume). Third operation (1608): the control system 2 may determine if the minimal airflow condition Q≥Qₘᵢₙᵢₘ is satisfied (where Q is a measure of the airflow in the enclosure and Qₘᵢₙᵢₘ is a minimum-flow-threshold). The three operations above can be performed sequentially in the order: first operation, second operation, third operation.

If any of the conditions above fails, the control system 2 may continue pumping with a higher than minimal value for the pumping power. A level of the pumping power may depend on the last failed condition, on the values of ΔP, on the value of the failed variable, and on other parameters of operation at the condition failure time (operation 1604).

Based on known or approximated relationship between the inside-outside differential pressure ΔP and the flow, Q=Q(ΔP), and based on the measured values of the differential pressure at time moments tₙ, ΔP(tₙ)=ΔPₙ, the control system 2 may determine the approximate total flown air volume during a time T, Qᵥ(T) over a large time interval, T=t_{N}_{₂}−t_{N}_{₁} as Qᵥ(T)≈Σ_{n=N}_{₁}^{N}^{₂}Q(ΔPₙ)δt, where δt is the time interval between two successive time steps, δt=tₙ₊₁−tₙ. In some applications, such as medical applications, T is one hour, with the standard setting for surgery applications that Qᵥ(T=1 hour)≥40 Vol_{encl}, where Vol_{encl} is the volume of the enclosure. The approximation of Qᵥ(T) can be further refined, taking into account that the flow resistance R_{flow} of the vent (or of the leaks) of the enclosure in the relation Q·R_{flow}=ΔP increases about quadratically with the flow.

At each point when a condition is satisfied, the control system 2 may learn the values of the control-parameters (e.g. pumping-power, valve status) and the measured physical parameters (e.g. pressure, temperatures, airflows) that satisfied the previous conditions and the dynamics of the pressurization (operation 1612). For example, when the enclosure system reaches a normal operation regime, the control system 2 may learn the temporal sequence of control parameters and physical parameters prior to achieving normal operation. In some cases, when the enclosure system is at a fault operation regime, the control system 2 may learn the temporal sequence of control parameters and physical parameters leading to the faulty operation. In some implementations, when the last condition satisfied is the airflow, airflow Q≥Qₘᵢₙᵢₘ, the values P, σ that satisfied the minimal airflow Qₘᵢₙᵢₘ is learned, moreover the dynamics of pressurization for reaching Qₘᵢₙᵢₘ, together with ambient conditions, noise level during the process, and consumed power during the process, are learned.

In some implementations, with reference to FIG. 16, a method for operating the enclosure-system may include comparing the differential-pressure with a pressure-threshold; generating airflow into the enclosure at an airflow-level higher than a regular-airflow-level if and when the differential-pressure is smaller than the pressure-threshold. The method may further include generating a pass-signal if and when the differential-pressure is larger than the pressure-threshold, determining the wall-straightness of the enclosure, and comparing the wall-straightness with a straightness-threshold. The method may further include generating more airflow into the enclosure at an airflow-rate higher than a maintenance-airflow-rate if and when the wall-straightness is smaller than the straightness-threshold and generating a pass-signal if and when the wall-straightness is larger than the straightness-threshold. The method may further include determining the airflow into the enclosure and comparing the airflow with an airflow-threshold and generating more airflow into the enclosure if and when the airflow is smaller than the airflow-threshold. The method may further include generating a pass signal if and when the airflow is larger than the airflow-threshold; determining and learning the values of the differential-pressures, the wall straightness, and the airflows leading to a desirable operation regime.

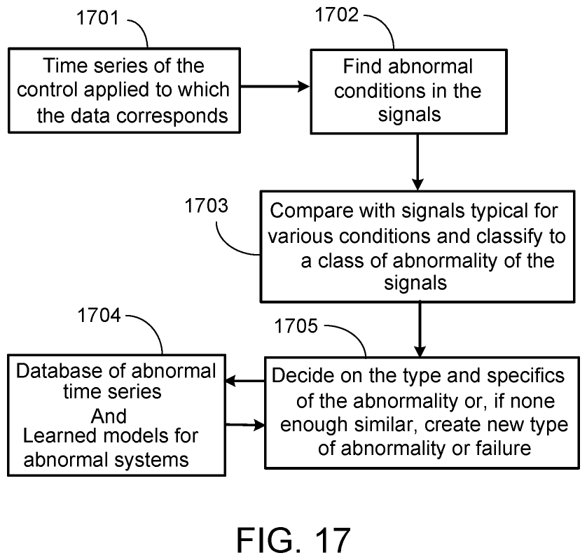

In some implementations, with reference to FIG. 17, a method for operating the enclosure system may include an operation 1701 in which the control system 2 acquires a time series of the control parameters leading to specific measured signals/data corresponding to the physical parameters of the enclosure system. The measured signals correspond to specific system status and/or specific operational regime. The operational regime may include one or more normal operation regime and one or more abnormality/faulty operation regimes.

The method may further include an operation 1702 in which the control system 2 uses the information acquired at operation 1701 to find abnormal conditions in the measured signals. The abnormal conditions are indicative of and associated with one or more of the abnormality/faulty operation regimes. The faulty operation regimes may be classified in a set of classes according to specifics of the abnormality/faulty operation regime, such as: a class corresponding to a leaky enclosure; a class corresponding to faulty connection between air source 3 and air duct 4; a class corresponding to reaching an enclosure contamination threshold; a class corresponding to a clogged air filter; a class corresponding to low inner pressure; and a class corresponding to above threshold vibrations.

The method may further include an operation 1703 in which the control system 2 compares the signals/data acquired at operations 1701 and 1702 (e.g., data indicative of the operational regime) with signals/data corresponding to a set of known abnormality/faulty operation regimes. Operation 1703 further includes classifying the signals/data acquired (and the corresponding operation regime) into the classes of abnormality/faulty operation regimes.

The method may further include an operation 1704 in which the control system 2 accesses a database including control parameters time series leading to specific abnormality/faulty operation regimes and including machine-learned models for the abnormality/faulty operation regimes.

The method may further include an operation 1705 in which the control system 2 uses the information generated at operations 1703 and 1704 to determine, for the signal/data acquired at 1701, the specific class of abnormality/faulty operation regime to which the signal/data corresponds. In case the signal/data acquired at 1701 does not correspond to a specific class, the control system 2 creates a new class of abnormality/faulty operation regime and associates the control parameters time series leading to the signals/data with this new class. The method may further include updating the databases of operation 1704 with the new class.

In some implementations, with reference to FIG. 17, a method for operating the enclosure system may include comparing a status of the enclosure system with a set of machine-learned system models that include fault operation models and proper operation models. The method may further include the control system 2 determining if the status of the enclosure system corresponds to a specific fault operation model. The control system 2 may be configured to generate classes of fault operation models (each class corresponding to a different type of faulty operation regimes) and classes of proper operation models (each class corresponding to a different class of proper (e.g., non-faulty) operation regimes). The method may further include determining if the system model corresponds to a class of fault operation models. If the system model corresponds to faulty operation and the system model does not correspond to any class of faulty operation models, then the control system 2 may create a new class of faulty operation models corresponding to the system model.

The learned temporal sequences of the control parameters and physical parameters are used to build machine learning models for the enclosure system. The learning is performed for the optimization of the control operation in relation with one or several specified criteria that may be associated with different phases. For example, when the enclosure 1 in flexible and should be inflated, attaining the inflation state, including or not the straightness of the walls, in a specified time, or as fast as possible may be the main optimization criteria for the first phase or for the first two phases. In contrast, attaining a specified average airflow during the first few minutes or even tens of minutes may be not essential as long as an average per hour airflow is obtained. Therefore, a state of reaching a specified airflow per hour may be obtained while seeking to minimize the power consumption or the average level of noise. Optimization of the operation under various optimization criteria without any fundamental change of the control system 2 or of its main features.

Learning of optimized control, according to a specified set of criteria, is performed by the control system 2 continuously. In some implementations, Bayesian neural networks, which are an established method for optimizing otherwise difficult to evaluate black-box systems, may be used. In some implementations, the learning system is based on a neural network inverse model, which can be suitable for extracting the model of complex systems and optimize their control or design. In some implementations, the control system can use a rule-based control with adjustable rules, where the rules are optimized during the learning of the system.

The implementations described in this disclosure beneficially allow an enclosure system to be controlled for optimizing various settings such as pressure and air flow. As described above and illustrated in the accompanying figures, the control system 2 may be configured to control and monitor the dynamics of inflation processes and the dynamic of enclosure pressurization during various inflation regimes. The inflation regimes includes stationary regimes during which the environmental parameters (e.g. pressures and airflows) of the enclosure system are stable/constant in time. The enclosure systems are generally used for their intended purpose during such stationary regimes. Such a stationary regime may be configured to maintain a specified minimal airflow through the enclosure while monitoring the enclosure and adapting to the circumstances of the controlled process. The control system may be configured to maintain a constant air pressure in an enclosure and at the same time support a circulation of the air through the enclosure.

The control system 2 may be configured to control airflow through enclosures under dynamic regimes. The enclosures may be flexible and inflatable. The enclosures may be rigid fixed volume enclosures. The control system 2 may be configured to first inflate an inflatable enclosure and, after the inflation state is achieved, to maintain a constant air pressure in the enclosure while supporting air circulation through the enclosure.

The control system 2 may be configured to determine if the enclosure is an inflatable enclosure or a fixed volume enclosure by analyzing the evolution/dynamics patterns of the physical parameters of the enclosure (e.g. evolution in time of airflows, pressures). The control system 2 may be configured to activate an alarm or alarms to warn users of abnormal inflation or other deviation from known inflation patterns.

The control system 2 may be configured to monitor and control the state of the walls of an inflatable enclosure during inflation and during the maintaining of a constant air pressure while air circulates through the enclosure. The control system 2 may be configured to control the enclosure according to the wall states.

The control system 2 may be configured to monitor and control the sound levels and vibrations levels generated during inflation and stationary states.

The control system 2 may be configured to learn models of behavior and to form knowledge about enclosures' inflation patterns. The control system 2 may be configured to recognize if a connected enclosure is of the type of a previously learned enclosure and further to use the enclosure model and knowledge to improve the pressure control in that type of enclosure.

The control system 2 may be configured to perform one or more of the following tasks: (i) provide purified air to an enclosure, (ii) control the inflation of a flexible enclosure and of fixed volume enclosures, (iii) control the pressures inside the enclosure while the air is circulated through the enclosure at a specified airflow rate, (iv) monitor the adequacy of the setting (mounting) of the enclosure system and the control system 2, (v) test and monitor the operation of power supplies included in the enclosure system, (vi) monitor the state of the air filtering element or elements, (vii) control the air flow through the enclosure during a normal operation regime at constant pressure, (viii) monitor the circulated air quality, (ix) generate a set of alarm signals when any inappropriate condition occurs, and (x) adapt the entire control, including the initial phases of inflation or pressurization, to the behavior of the enclosure and of the entire pressure maintenance process, including to the noise and vibrations generated for improving the control.

Furthermore, the control system 2 may be configured to learn about the control process and improve the control by adaptation to a specific type of enclosure previously learned. The control system 2 may be configured to: determine situations when the use of the enclosure system poses a threat of contamination by unpurified air; remember that such conditions occurred; and generate alarm signals when such situations occur so as to prevent the further use of the contaminated system.

The control system 2 may be configured to perform one or several tasks, including: a) acquire data from multiple sensors and measuring devices; b) monitor the deployment of the enclosure system, determine the contamination degree of the enclosure during deployment, and store the acquired information; c) monitor the state of the enclosure, including the state of its walls for flexible and inflatable enclosures d) determine a system models for the enclosure system and store the system models; e) determine the level of depletion of the power supply when the power supply has a limited capacity; e) display information about the operation and provide guidance; and f) communicate with other systems and transfers data and learned information to and from them.

The control system 2 may include a set of sensors. The sensors may be disposed inside the enclosure or on the enclosure wall; the sensors may be included into and form an integral part with the enclosure walls. The control system 2 may include sensors remotely connected to the enclosure through air pressure sampling tubes, sensors gathering data about the enclosure from a distance, or a combination thereof.

The control system 2 may include one or more components configured to transmit the data from the sensors to the electronic system for data acquisition, process the data, analyze the data, store the data, with the aim of controlling and monitoring the process and the enclosure, and of machine learning from data. The data processing may include decision making on the manner of controlling the air pressure and airflow, information communication, data and information displaying, and alarm generation. The control system 2 may uses specific algorithms performing machine learning towards improving the control and for adapting to the circumstances of pressure and airflow control.

While the control system 2 may be used with inflatable enclosures having air circulation, it should be understood that the various changes, substitutions, and alterations could be made hereto without departing from the spirit and scope of the present disclosure. In some implementations, fixed volume enclosures, flexible enclosures, sealed enclosures, and enclosures with vents and air circulation may be used.

The implementations described above may be embodied in many different forms and should not be construed as limited to the implementations explicitly described herein. Various changes, modifications, and equivalents of the systems, apparatuses and/or methods described herein will suggest themselves to those of ordinary skill in the art. Descriptions of well-known functions and structures are omitted to enhance clarity and conciseness.

It should be understood that one or more components of the control system 2 can be implemented remotely from the enclosure 1 and may be connected to the enclosure 1 through a wired or wireless communication network. For example, a processor can be located remotely from the enclosure 1, can receive data from one or more sensors, and can generate and transmit instructions to one or more components coupled to the enclosure 1 to maintain one or more conditions, e.g., pressure, within the enclosure 1. Various suitable networks can be used to facilitate communications with the control system 2. For example, long distance and/or short distance wireless networks such as Wi-Fi networks, cellular networks, or Bluetooth piconets can be used to facilitate communications between various components of the control system or between the control system 2 and other devices.

The described systems, methods, and techniques may be implemented using digital electronic circuitry, computer hardware, firmware, software, or in combinations of these elements. Apparatus implementing these techniques may include appropriate input and output devices, a computer processor, and a computer program product tangibly embodied in a machine-readable storage device for execution by a programmable processor. A process implementing these techniques may be performed by a programmable processor executing a program of instructions to perform desired functions by operating on input data and generating appropriate output. The program may be implemented using one or more computer programs or non-transitory computer-readable storage media that includes instructions that are executable on a programmable system including at least one programmable processor coupled to receive data and instructions from, and to transmit data and instructions to, a data storage system, at least one input device, and at least one output device. Each computer program may be implemented in a high-level procedural or object-oriented programming language, or in assembly or machine language if desired; and in any case, the language may be a compiled or interpreted language. Suitable processors include, by way of example, both general and special purpose microprocessors. Generally, a processor will receive instructions and data from a read-only memory and/or a random access memory. Storage devices suitable for tangibly embodying computer program instructions and data include all forms of non-volatile memory, including by way of example, semiconductor memory devices, such as Erasable Programmable Read-Only Memory (EPROM), Electrically Erasable Programmable Read-Only Memory (EEPROM), and flash memory devices; magnetic disks such as internal hard disks and removable disks; magneto-optical disks; and Compact Disc Read-Only Memory (CD-ROM). Any of the foregoing may be supplemented by, or incorporated in, specially designed ASICs (application-specific integrated circuits).

Computer-readable medium may be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program, also known as a program, software, software application, script, plug-in, or code, may be written in any form of programming language, including compiled or interpreted languages, and it may be deployed in any form, including as a standalone program or as a module, component, subroutine, or other unit suitable for use in a computing environment. A computer program does not necessarily correspond to a file in a file system. A program may be stored in a portion of a file that holds other programs or data in a single file dedicated to the program in question, or in multiple coordinated files. A computer program may be executed on one computer or on multiple computers that are located at one site or distributed across multiple sites and interconnected by a communication network.

The processes and logic flows described in this specification may be performed by one or more programmable processors executing one or more computer programs to perform actions by operating on input data and generating output. The processes and logic flows may also be performed by, and apparatus may also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both.

Elements of a computer may include a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer may not have such devices. Moreover, a computer may be embedded in another device, e.g., a tablet computer, a mobile telephone, a personal digital assistant (PDA), a mobile audio player, a Global Positioning System (GPS) receiver, to name just a few. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory may be supplemented by, or incorporated in, special purpose logic circuitry.

While this specification contains many specifics, these should not be construed as limitations on the scope of the disclosure or of what may be claimed. Certain features that are described in this specification in the context of separate implementations may also be combined. Conversely, various features that are described in the context of a single implementation may also be implemented in multiple separate implementations or in any suitable sub-combination. Moreover, although features may be described above as acting in certain combinations and may even be claimed as such, one or more features from a claimed combination may, in some cases, be excised from the combination, and the claimed combination may be directed to a sub-combination or variation of a sub-combination. For example, although the mapping operation is described as a series of discrete operations, the various operations may be divided into additional operations, combined into fewer operations, varied in order of execution, or eliminated, depending on the desired implementation.

Similarly, the separation of various system components in the implementations described above should not be understood as requiring such separation in all implementations, and it should be understood that the described program components and systems may generally be integrated together in a single software product or packaged into multiple software products. For example, although some operations are described as being performed by a processing server, one of more of the operations may be performed by the smart meter or other network components.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. The term “and/or” should be construed in this manner. Additionally, the terms “about,” “substantially,” or “approximately” should be interpreted to mean a value within 10% of an actual value, for example, values like 3 mm or 100% (percent).

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms “first,” “second,” “third,” etc., connote a specific number of elements, these terms should not be understood to connote a specific number of elements.

Although embodiments of the present disclosure have been described in detail, it should be understood that the various changes, substitutions, and alterations could be made hereto without departing from the spirit and scope of the present disclosure. Other implementations are within the scope of the following claims. For example, the actions recited in the claims may be performed in a different order and still achieve desirable results.

1. An enclosure system comprising:
one or more enclosure walls comprising flexible materials and at least one transparent section, the one or more enclosure walls being walls of an enclosure;
an air vent having a variable pneumatic resistance; and
a control system configured to control an environment within the enclosure, the control system comprising:
an air source configured to provide air within the enclosure;
one or more sensors comprising wall state sensors coupled to the one or more enclosure walls and configured to obtain wall state data indicative of a straightness level of the one or more enclosure walls and an inflation level of the enclosure,
a processor configured to:
receive the wall state data from the wall state sensors;
determine the inflation level of the enclosure based on the wall state data;
control an airflow provided by the air source and through the air vent;
control a pressure inside the enclosure and the airflow into the enclosure such that the straightness level satisfies a straightness threshold of the enclosure.

2. The enclosure system of claim 1, wherein the one or more sensors comprise:
one or more pressure sensors configured to measure differential pressures between an interior of the enclosure and an exterior of the enclosure;
wherein the processor is further configured to:
receive pressure data from the one or more pressure sensors, the pressure data being indicative of the differential pressures and a dynamic evolution of the differential pressures,
determine the inflation level of the enclosure based on the pressure data and the wall state data; and
control the pressure inside the enclosure and the airflow into the enclosure such that the pressure inside the enclosure is in a predetermined pressure range and the straightness level of the one or more enclosure walls is above a minimum straightness threshold;
wherein a maximum value of the pressure inside the enclosure is less than a pressure level associated with a patient safety threshold limit.

3. The enclosure system of claim 2, wherein:
at least one of the one or more pressure sensors is attached to one or more enclosure walls and is coupled to a first measuring surface disposed inside the enclosure and a second measuring surface disposed outside the enclosure to measure an ambient pressure; and/or
at least one of the one or more pressure sensors is coupled to pressure tubes penetrating the one or more enclosure walls.

4. The enclosure system of claim 2, wherein:
the one or more sensors comprise airflow sensors configured to detect airflow through one or more portions of the enclosure; and
the processor is configured to control the pressure inside the enclosure and the airflow into the enclosure such that the pressure inside the enclosure is in the predetermined pressure range, the straightness level of the one or more enclosure walls is above the minimum straightness threshold, and the airflow into the enclosure is within a particular airflow range.

5. The enclosure system of claim 1, wherein at least one of the wall state sensors is coupled to a portion of the one or more enclosure walls and comprises:
a flexible foil mechanically connected to the portion of the one or more enclosure walls; and
a pressure sensing device disposed between the portion of the one or more enclosure walls and the flexible foil,
wherein the flexible foil is configured to apply a compression force on the pressure sensing device when the wall is straightened; and
wherein the pressure sensing device is configured to measure a pressure between a surface of the enclosure and a surface of the flexible foil;
wherein the processor is configured to receive the wall state data from the pressure sensing device and to determine the inflation level of the enclosure based on the walls state data.

6. The enclosure system of claim 5, wherein the pressure sensing device comprises one or more of the following:
a piezoelectric device; and
a capacitive sensor configured to determine a pressure force applied to the pressure sensing device based on a change in a spacing between capacitor plates of the capacitive sensor.

7. The enclosure system of claim 1, wherein the wall state sensor comprises one or more of the following:
a strain gauge connected to an inner surface or an outer surface of the enclosure and configured to deform together with the one or more enclosure walls;
an interdigital capacitor coupled to the one or more enclosure walls, the interdigital capacitor being configured to deform together with the one or more enclosure walls, the deformation of the interdigital capacitor corresponding to a change in a capacitance of the interdigital capacitor; and
a camera configured to obtain an image of the enclosure wall, to provide the image to the processor for determining the straightness level based on the image.

8. The enclosure system of claim 1, wherein the wall state sensor comprises an optical fiber, a light source, and a fiber detector, wherein the optical fiber is attached to the one or more enclosure walls and configured to deform together with the one or more enclosure walls, the deformation of the optical fiber corresponding to a change in a signal detected by the fiber detector.

9. The enclosure system of claim 4, wherein the air source further comprises:
an air source case comprising:
an air pump; and
a filter coupled to the air pump and configured to filter the airflow generated by the air pump; and
an air duct connecting the air source case to the enclosure and configured to provide filtered air from the air source to the enclosure, the air duct comprising one or more of:
at least one end detachable from the enclosure or from the air source case;
a pressure sampling tube configured to detect a pressure level in the air duct;
a section extending outside the enclosure;
one or more sections inside the enclosure; and
one or more valves for preventing backflow into the case.

10. The enclosure system of claim 9, wherein the air source case further comprises:
one or more airflow separators disposed in proximity of the filter and configured to separate the airflow;
one or more filter sensors configured to detect at least one of air flows, pressures, or both air flows and pressures over individual filter areas; and
differences between air flows, pressures, or both air flows and pressures over adjacent filter areas,
wherein the processor is configured to receive filter sensor data from the filter sensors, to determine a filter status, and to generate a message indicative of the filter status, and display a message indicative of the filter status.

11. The enclosure system of claim 9, comprising an adaptor configured to connect the air duct to the enclosure, the adaptor comprising connection sensors configured to detect whether the adaptor is connected to the air duct.

12. The enclosure system of claim 9, further comprising:
vibration sensors configured to sense vibrations of the enclosure system; and
sound sensors configured to sense sound generated by the enclosure system,
wherein the processor is configured to control the airflow provided by the air pump based on data received from at least one of the vibration sensors and the sound sensors to attain an operational state in which the vibrations are lower than a vibration threshold and the sound is lower than a sound threshold.

13. The enclosure system of claim 1, wherein the air vent is partially open to permit the air to be continually circulated throughout the enclosure.

14. The enclosure system of claim 1, wherein at least one of the air vents comprises a pressure sampling tube extending from the air vent to a region within the enclosure, the pressure sampling tube being connected to a pressure sensor configured to detect a pressure in the region within the enclosure.

15. The enclosure system of claim 1, wherein the processor is configured to determine a status of the enclosure system and to generate an alarm signal in response to determining that the status of the enclosure system is indicative of a contamination threat to the enclosure.

16. The enclosure system of claim 1, further comprising one or more strip antennas disposed on the one or more enclosure walls, wherein the strip antennas are configured to wirelessly transmit or receive data from the one or more strip antennas to the processor.

17. An enclosure system comprising:
one or more enclosure walls made of flexible materials and at least one transparent section, the one or more enclosure walls being walls of an enclosure; and
a control system configured to control an environment inside the enclosure, the control system comprising:
an air source configured to provide air within the enclosure;
one or more sensors comprising one or more pressure sensors configured to provide data for determining differential pressures between an interior of the enclosure and an exterior of the enclosure;
a processor configured to:
receive pressure data from the one or more pressure sensors indicative of the differential pressures and a dynamic evolution of the differential pressures,
determine an inflation level of the enclosure based on the pressure data; and
control a pressure inside the enclosure and an airflow through the enclosure such that an inner enclosure pressure is within a particular pressure range and the airflow through the enclosure is within a particular airflow range;
wherein a maximum value of the pressure inside the enclosure is less than a pressure level associated with a patient safety threshold limit.

18. The enclosure system of claim 17, wherein:
at least one of the one or more pressure sensors is attached to the one or more enclosure walls and is coupled to a first measuring surface disposed inside the enclosure and a second measuring surface disposed outside the enclosure to measure an ambient pressure; and/or
at least one of the one or more pressure sensors is coupled to pressure tubes penetrating the one or more enclosure walls.

19. The enclosure system of claim 17, wherein the one or more sensors comprise:
one or more wall state sensors coupled to the one or more enclosure walls and configured to obtain wall state data indicative of a straightness level of the one or more enclosure wall and the inflation level of the enclosure,
wherein the processor is configured to:
receive the wall state data from the one or more wall state sensors;
determine the inflation level of the enclosure based on the wall state data and the pressure data to;
control the airflow provided by the air source and through air vents; and
control the pressure inside the enclosure and the airflow into the enclosure such that the pressure inside the enclosure is in a predetermined pressure range and the straightness level of the one or more enclosure walls is above a minimum straightness threshold.

20. The enclosure system of claim 19, wherein:
the one or more sensors comprise airflow sensors configured to detect the airflow through one or more portions of the enclosure; and
the processor is configured to control the pressure inside the enclosure and the airflow into the enclosure such that the pressure inside the enclosure is in the predetermined pressure range, maintain the straightness level of the one or more enclosure walls is above the minimum straightness threshold, and the airflow through the enclosure is within a particular airflow range.

21. A method of operating an enclosure system comprising an enclosure and an air pump, the method comprising:
determining a differential pressure between an interior of an enclosure and an exterior of the enclosure; and
controlling an output level of an air pump to an airflow output value based on the differential pressure,
wherein the airflow output value is:
in a first airflow range when the differential pressure is smaller than a first pressure value;
in a second airflow range when the differential pressure is larger than the first pressure value and smaller than a second pressure value;
in a third airflow range when the differential pressure is larger than the second pressure value.

22. The method of claim 21, wherein:
the first airflow range corresponds to a first level of airflow generated by the air pump;
the second airflow range corresponds to a second level of airflow generated by the air pump;
the third airflow range corresponds to a third level of airflow generated by the air pump;
the first level of air flow is greater than the second level of air flow; and
the second level of air flow is greater than the third level of air flow.

23. The method of claim 21, wherein the airflow output value is determined based on a set of adjustable parameters comprising one or more of: the first pressure value, the second pressure value, the first airflow range, the second airflow range, and the third airflow range.

24. The method of claim 23, wherein the adjustable parameters are selected to inflate the enclosure at an inflation rate greater than a first inflation rate.

25. The method of claim 21, the method comprising:
comparing the differential pressure with a pressure threshold;
providing an airflow into the enclosure at the airflow output value that has an airflow output level higher than a first airflow threshold in response to the differential pressure being smaller than the pressure threshold;
generating a first pass signal in response to the differential pressure being larger than the pressure threshold;
determining a straightness level of the enclosure;
comparing the straightness level with a straightness threshold;
providing the airflow at a second airflow output value greater than the airflow output value into the enclosure in response to determining that the straightness level is less than the straightness threshold;
generating a second pass signal in response to determining that the straightness level is greater than the straightness threshold;
comparing the airflow with a second airflow threshold;
providing the airflow into the enclosure at a third airflow output value in response to determining that the airflow is smaller than the second airflow threshold;
generating a third pass signal in response to determining that the airflow is larger than the second airflow threshold; and
determining and learning values of the differential pressure, the straightness level of the enclosure, and the airflow leading to a particular operation regime.

26. The method of claim 21, the method comprising:
periodically determining the differential pressure;
periodically comparing the determined differential pressure with a pressure threshold;
controlling the enclosure system to operate in a first operation regime in response to determining that the differential pressure is greater than the pressure threshold;
triggering a failure alarm in response to determining that the differential pressure is less than the pressure threshold after a certain time period has elapsed from a time that inflation of the enclosure started.

27. The method of claim 21, the method comprising:
obtaining, periodically, measurements of values of one or more physical parameters, the physical parameters comprising a pressure of a portion of the enclosure, an airflow in the portion of the enclosure, a wall status in the portion of the enclosure, a vibration in the portion of the enclosure, and a sound generated by the enclosure system;
generating one or more physical models for the enclosure system describing a vibrational behavior of the enclosure system based on the values of the one or more physical parameters; and
setting, periodically, control parameters for the enclosure system, the control parameters comprising one or more of valve parameters and pump parameters;
determining a status of the enclosure system, based on the physical parameters and the control parameters;
determining whether the status of the enclosure system corresponds to a faulty operation mode or a non-faulty operation mode; and
in response to determining that the status of the enclosure system corresponds to the faulty operation mode, determining values for the control parameters to operate the enclosure system in the non-faulty operation mode,
wherein the values of the control parameters are determined based on the status of the enclosure system, the one or more physical models of the enclosure system, and one or more machine-learned system models associated with the enclosure system.

28. The method of claim 27, comprising:
generating a system model based on the status of the enclosure system, the one or more physical models of the enclosure system, and the one or more machine-learned system models associated with the enclosure system; and
updating the one or more machine-learned system models using the generated system model.

29. The method of claim 28, comprising:
comparing the status of the enclosure system with the one or more machine-learned system models, the one or more machine learned system models comprising a fault operation model and a non-faulty operation model to determine whether the status of the enclosure system corresponds to the fault operation model;
generating classes of fault operation models and classes of proper operation models;
determining whether the system model corresponds to a class of fault operation models; and
generating a second class of faulty operation models comprising the system model in response to determining that the system model corresponds to the class of faulty operation.

30. The method of claim 21, the method comprising:
performing a pre-operation test;
performing a first enclosure inflation, a first pressurization, and a first functionality test of the enclosure;
receiving sensor data from one or more sensors of the enclosure system;
determining one or more pneumatic-parameters associated with the enclosure system based on the sensor data;
determining whether the enclosure system is in a non-faulty pressure flow regime;
performing one or more functionality tests;
generating a pneumatic model of the enclosure system for system dynamics and sequences of control parameters;
performing machine learning for the system dynamics and generating machine-learned parameters; and
setting the control parameters based on the sensor data, the one or more functionality tests, and the machine-learned parameters.
